(19) United States
(12) Patent Application Publication
(10) Pub. No.: US 2010/0015239 A1
(43) Pub. Date: Jan. 21, 2010

(54) Orally Disintegrating Solid Pharmaceutical Dosage Forms Comprising Delayed-Release Lansoprazole and Methods of Making and Using the Same

(76) Inventors: Salah U. AHMED, New City, NY (US); Sudhir R. Gorukanti, Harriman, NY (US); Venkatesh Naini, Parsippany, NJ (US)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 12/504,244

(22) Filed: Jul. 16, 2009

Related U.S. Application Data
(60) Provisional application No. 61/129,757, filed on Jul. 17, 2008.

(51) Int. Cl. A61K 9/16; A61K 31/4439; A61P 1/00

(52) U.S. Cl. 424/497; 514/338; 424/490

(57) ABSTRACT

The present invention relates to non-effervescent, orally disintegrating solid pharmaceutical dosage forms comprising delayed-release lansoprazole, processes for preparing the dosage forms, and methods for treating one or more conditions with the dosage forms.

### CROSS-REFERENCE TO RELATED APPLICATIONS

This applications claims the benefit of the filing date of U.S. application Ser. No. 61/129,757, filed Jul. 17, 2008, which is incorporated herein by reference in its entirety.

### BACKGROUND OF THE INVENTION

1. Field of the Invention

The present invention is directed to non-effervescent, orally disintegrating solid pharmaceutical dosage forms comprising delayed-release lansoprazole, processes to prepare the dosage forms, and methods of using the dosage forms to treat conditions in subjects in need thereof.

2. Background

Lansoprazole is a member of a class of anti-secretory compounds, the substituted benzimidazoles, which exert a therapeutic effect by selectively inhibiting the (H⁺, K⁺)-ATPase enzyme system at the secretory surface of gastric parietal cells, thus interfering with the stomach's production of acid.

Lansoprazole is indicated for use in treating a wide variety of conditions, including: the short-term treatment of healing and symptom relief of active duodenal ulcer; the healing of duodenal ulcers; the short-term treatment, healing and symptom relief of active benign gastric ulcers; the treatment of NSAID-associated gastric ulcer in patients who continue NSAID use; reducing the risk of NSAID-associated gastric ulcers in patients with a history of a documented gastric ulcer who require the use of an NSAID; the treatment of heartburn and other symptoms associated with gastro-esophageal reflux disease (“GERD”); the short-term treatment, healing, symptom relief and maintained healing of all grades of erosive esophagitis; and the long-term treatment of pathological hypersecretory conditions, including Zollinger-Ellison syndrome.

While lansoprazole effectively treats a wide variety of gastrointestinal disorders, it is subject to pH-dependent degradation, and therefore should be administered in a manner that prevents its exposure to the acidic environment of the stomach. To circumvent this problem, lansoprazole is presently available in delayed-release capsule, oral suspension, and orally disintegrating tablet dosage forms that contain a 15 mg or 30 mg dosage of lansoprazole (e.g., PREVACID®, TAP Pharmaceuticals Inc., Lake Forest, Ill.).

Gastric conditions, in particular, can affect or disable a subject at inopportune times such as while traveling, or after a meal, when a conventional solid pharmaceutical dosage form may not be easily administered. Additionally, a large percentage of the adult population suffers from dysphagia and therefore may have difficulty swallowing solid pharmaceutical dosage forms even with water. Thus, a non-effervescent, orally disintegrating dosage form capable of administration without water is desirable both for convenience and due to its potential widespread acceptance by patients.

However, typical delayed-release delivery systems such as osmotic pumps and enteric coatings can be difficult to incorporate into a non-effervescent, orally disintegrating solid pharmaceutical dosage form while maintaining acceptable taste and mouth feel. Such organoleptic properties are highly important for a non-effervescent, orally disintegrating dosage form, because the experience of any grittiness upon administration will minimize patient acceptance and compliance with any dosage regimen. As a general rule, the incorporation in orally disintegrating dosage forms of particles having a size of more than 400 μm has been shown to lead to unacceptable grittiness and poor mouth feel in subjects. See, e.g., U.S. Pat. No. 6,328,994 B1.

Additionally, orally disintegrating dosage forms have a tendency to break apart during storage, transport, or otherwise prior to administration. This is usually because properties that can provide for rapid orally disintegrating action can also lead to a high friability. Thus, there is a need to balance numerous competing properties, such as providing orally disintegrating dosage forms having a suitable hardness and friability that also maintain a disintegration rate of about 60 seconds or less and have acceptable organoleptic properties. For purposes of high-volume manufacturing, it is also desirable that the dosage forms are manufacturable using a traditional tableting apparatus, and therefore, that the precursor mixture to the dosage forms be capable of undergoing compression at conventional tableting pressures.

### BRIEF SUMMARY OF THE INVENTION

The present invention is directed to a non-effervescent, orally disintegrating solid pharmaceutical dosage form, the dosage form comprising a compacted granulate-bead mixture, the granulate-bead mixture comprising:

a bead portion comprising a lansoprazole spheroid having an aspect ratio of about 0.75 to about 1.3 and having an enteric coating layer and a deformable coating layer thereon, wherein the deformable coating layer is about 10% to about 30% by weight of the bead portion and the bead portion has a D₅₀ of 400 μm or greater; and
a granulate portion comprising a disintegrant in a concentration of about 20% to about 60% by weight of the granulate,
wherein the bead portion and the granulate portion are present in a ratio of about 1:3 to about 3:1 and wherein upon oral administration without water the dosage form disintegrates in about 60 seconds or less.

The present invention is also directed to a process to prepare a non-effervescent, orally disintegrating solid pharmaceutical dosage form, the process comprising:

mixing a bead portion comprising lansoprazole spheroids having an aspect ratio of about 0.75 to about 1.3 and having an enteric coating layer and a deformable coating layer thereon, wherein the deformable coating layer is about 10% to about 30% by weight of the bead portion and the bead portion has a D₅₀ of 400 μm or greater with a granulate portion comprising a disintegrant in a concentration of about 20% to about 60% by weight of the granulate, wherein the bead portion and the granulate portion are present in a ratio of about 1:3 to about 3:1, to form a substantially homogeneous granulate-bead mixture; and
compacting the granulate-bead mixture at a pressure of about 50 kN or less to provide the non-effervescent, orally disintegrating solid pharmaceutical dosage form, wherein upon oral administration without water the solid pharmaceutical dosage form disintegrates in about 60 seconds or less.

The present invention is also directed to a product prepared by the process.

The present invention is also directed to a method of treating gastro-esophageal reflux disease (“GERD”) or a symptom thereof in a subject in need thereof, the method comprising administering a non-effervescent, orally disintegrating solid pharmaceutical dosage form, the dosage form comprising a compacted granulate-bead mixture comprising:

a bead portion comprising a lansoprazole spheroid having an aspect ratio of about 0.75 to about 1.3 and having an enteric coating layer and a deformable coating layer thereon, wherein the deformable coating layer is about 10% to about 30% by weight of the bead portion and the bead portion has a D₅₀ of 400 μm or greater; and
a granulate portion comprising a disintegrant in a concentration of about 20% to about 60% by weight of the granulate,
wherein the bead portion and the granulate portion are present in a ratio of about 1:3 to about 3:1 and wherein upon oral administration without water the dosage form disintegrates in about 60 seconds or less.

In some embodiments, the granulate portion comprises directly compressible mannitol in a concentration of about 1% to about 20% by weight of the granulate, and microcrystalline cellulose in a concentration of about 10% to about 40% by weight of the granulate. In some embodiments, the granulate portion comprises directly compressible mannitol in a concentration of about 1% to about 10% by weight of the granulate. In some embodiments, the granulate portion comprises microcrystalline cellulose in a concentration of about 20% to about 40% by weight of the granulate.

In some embodiments, the granulate portion comprises crospovidone in a concentration of about 20% to about 60% by weight of the granulate.

In some embodiments, the granulate portion comprises colloidal silicon dioxide in a concentration of about 10% or less by weight.

In some embodiments, the granulate portion comprises a lubricant in a concentration of about 10% or less by weight.

In some embodiments, the bead portion and the granulate portion are present in a ratio of about 1:2 to about 2:1 by weight.

In some embodiments, the bead portion has a D₅₀ of 400 μm to about 550 μm. In some embodiments, the bead portion has a D₁₀ of about 350 μm or greater. In some embodiments, the bead portion has a D₉₀ of about 800 μm or less.

In some embodiments, the granulate portion has a D₅₀ of about 50 μm to about 400 μm.

In some embodiments, the bead portion comprises a lansoprazole spheroid having a protective coating layer thereon, wherein the lansoprazole spheroid and protective coating layer comprise a hydroxypropyl cellulose polymer having a molecular weight of about 95,000 Da or less.

In some embodiments, the deformable coating layer comprises polyethylene glycol, microcrystalline cellulose, and a glidant.

In some embodiments, the deformable coating layer is about 10% to about 30% by weight of the bead portion.

In some embodiments, a surface of the deformable coating layer has a surface roughness of about 50 nm to about 10 μm.

In some embodiments, the lansoprazole spheroids have an acid resistance of about 20% or less, as tested using a USP Type II Paddle Apparatus containing 475 mL of 0.1 N HCl at a paddle speed of 75 rpm.

In some embodiments, the bead portion has a crush strength of about 300 g or more. In some embodiments, the solid pharmaceutical dosage form has a hardness of about 1 kp to about 5 kp.

### BRIEF DESCRIPTION OF THE FIGURES

FIG. 1 is a cross-sectional schematic representation of a bead portion of a solid pharmaceutical dosage form of the present invention.

FIG. 2 is a process flow chart representing a process for preparing lansoprazole spheroids of the present invention.

FIG. 3 is a process flow chart representing a process for preparing a bead portion of the present invention.

FIG. 4 is a digital image of a scanning electron micrograph of an enteric layer coated bead portion of the present invention.

FIG. 5 is a digital image of a scanning electron micrograph of a final coated bead portion of the present invention.

FIG. 6 is a digital image of a scanning electron micrograph of a cross-sectional cutaway of a final bead portion of the present invention.

FIG. 7 is a process flow chart representing a process for preparing a non-effervescent, orally disintegrating solid pharmaceutical dosage form of the present invention.

### DETAILED DESCRIPTION OF THE INVENTION

The present invention is directed to orally disintegrating solid pharmaceutical dosage forms comprising delayed-release lansoprazole. The chemical name for lansoprazole is 2-[[[3-methyl-4-(2,2,2-trifluoroethoxy)-2-pyridyl]-methyl]sulfinyl]benzimidazole. As used herein, “lansoprazole” refers to salts, hydrates, solvates, and the like of lansoprazole and combinations thereof. Lansoprazole has the following structure:

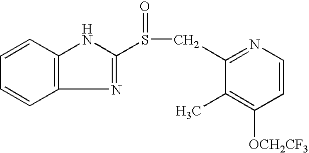

Lansoprazole contains a chiral atom, and thus, as used herein, “lansoprazole” refers to the levorotatory enantiomer, dextrorotatory enantiomer, racemic mixtures of these enantiomers, and combinations thereof. Accordingly, as used herein, the term “lansoprazole” contemplates all such forms, as well as prodrugs, esters, and any other analogs of the above structure. Lansoprazole for use with the present invention can have a crystalline, amorphous and/or particulate morphology, and combinations thereof. Lansoprazole for use with the present invention can also include all polymorphic crystalline forms of lansoprazole, and any complexes between lansoprazole and a second species, a moiety, or the like.

In some embodiments, lansoprazole for use with the present invention is micronized, having a D₅₀ of about 5 μm or less, a D₁₀ of about 2 μm or less, and a D₉₀ of about 7 μm or less. In some embodiments, lansoprazole for use with the present invention has a surface area of about 2.2 m²/g or more, or about 2.25 m²/g or more.

Throughout the present disclosure, all expressions of percentage, ratio, corporation, and the like are “by weight” unless otherwise indicated. As used herein, “by weight” is synonymous with the term “by mass,” and indicates that a ratio or percentage defined herein is done according to weight rather than volume, thickness, or some other measure.

As used herein, the term “about,” when used in conjunction with a percentage or other numerical amount, means plus or minus 10% of that percentage or other numerical amount. For example, the term “about 80%” would encompass 80% plus or minus 8%.

### Orally Disintegrating Solid Pharmaceutical Dosage Forms

The present invention is directed to a non-effervescent, orally disintegrating solid pharmaceutical dosage form, the dosage form comprising a compacted granulate-bead mixture, the granulate-bead mixture comprising:

a bead portion comprising a lansoprazole spheroid having an aspect ratio of about 0.75 to about 1.3 and having an enteric coating layer and a deformable coating layer thereon, wherein the bead portion has a D₅₀ of 400 μm or greater, wherein the deformable coating layer is about 10% to about 30% by weight of the bead portion; and
a granulate portion comprising a disintegrant in a concentration of about 20% to about 60% by weight of the granulate,
wherein the bead portion and the granulate portion are present in a ratio of about 1:3 to about 3:1 and wherein upon oral administration without water the dosage form disintegrates in about 60 seconds or less.

The pharmaceutical dosage forms of the present invention provide safe and effective absorption of lansoprazole, as well as bioavailability and dissolution of lansoprazole equivalent to that achieved using other traditional solid pharmaceutical dosage forms.

As used herein, a “unit dosage” refers to an amount of the pharmaceutical composition that is individually administered. Unit dosage amounts of lansoprazole include, but are not limited to, 15 mg and 30 mg, or a multiple thereof.

As used herein, “composition” and “mixture” are used interchangeably and refer to a combination of two or more materials, substances, excipients, portions, and the like. In some embodiments, a mixture and/or a composition are substantially homogeneous. For example, a granulate-bead mixture comprises a granulate portion and a bead portion, the mixture of which is a substantially uniform distribution of beads within the granulate.

As used herein, “homogeneous” refers to mixtures, compositions and/or layers of the present invention that have a uniform distribution of ingredients throughout the layer, mixture and/or composition. Homogeneity is synonymous with uniformity and can refer to intra-sample uniformity, batch-to-batch uniformity, run-to-run uniformity, and/or dosage form-to-dosage form uniformity. For example, intra-sample uniformity can be determined by analyzing a first portion of a sample, mixture, or composition and comparing this with a second portion of the same sample, mixture, or composition. Typical deviations of a composition (e.g., variation in the percentage by weight of excipients and the like) of a substantially homogeneous mixture are about 10% or less, about 5% or less, about 3% or less, about 2% or less, about 1% or less, or within experimental error.

The orally disintegrating dosage forms of the present invention are non-effervescent. As used herein, “non-effervescent” refers to dosage forms lacking a combination of acid and base components, which upon contacting an aqueous medium such as water or saliva, react with each other to generate a gas. Thus, the dosage forms of the present invention do not rely on an acid-base reaction for disintegration. More generally, the non-effervescent dosage forms of the present invention do not rely on in situ gas generation for disintegration.

As used herein, “orally disintegrating” refers to the loss of structural integrity by the dosage forms upon their placement in the buccal cavity of a subject without the need for water to be administered to the subject. In some embodiments, the loss of structural integrity results in the formation of a viscous, semi-solid and/or liquid composition comprising suspended particulates that can be easily swallowed by a subject. The solid pharmaceutical dosage forms of the present invention are designed to disintegrate or dissolve rapidly on contact with saliva, thus eliminating the need for chewing the dosage form, swallowing the dosage form intact, or taking the dosage form with water.

As used herein, the term “disintegration” refers to the loss of structural integrity of the dosage forms of the present invention to form viscous, semi-solid and/or liquid compositions comprising granules, aggregates and/or particles, as is generally described in Remington: The Science and Practice of Pharmacy, 21st Ed., Lippincott Williams & Wilkins, Baltimore, Md. (2003), which is incorporated herein by reference in its entirety.

Additionally, the Food and Drug Administration (“the FDA”) provides guidance for industry relating to orally disintegrating dosage forms, as described in Draft Guidance for Industry: Orally Disintegrating Tablets, U.S. Department of Health and Human Services, Food and Drug Administration, Center for Drug Evaluation and Research (CDER), Rockville, Md. (April 2007), which is incorporated herein by reference in its entirety.

In some embodiments, “orally disintegrating” additionally refers to the ability of the solid pharmaceutical dosage forms of the present invention to disintegrate in saliva without effervescence in a time of about 60 seconds or less, about 45 seconds or less, about 30 seconds or less, about 20 seconds or less, about 15 seconds or less, about 10 seconds or less, or about 5 seconds or less without administering the dosage forms with administration or intake of water. In some embodiments, the orally disintegrating dosage forms of the present invention disintegrate without effervescence in the buccal cavity without additional water in a period of time of about 5 seconds to about 30 seconds, about 5 seconds to about 20 seconds, or about 5 seconds to about 15 seconds.

As used herein, a “solid pharmaceutical dosage form” or “solid dosage form” refer to a dosage form comprising a compacted granulate-bead mixture. The solid pharmaceutical dosage forms can be in the form of tablets, caplets, wafers, troches, and the like. In some embodiments, the solid pharmaceutical dosage forms of the present invention are orally disintegrating tablets. As used herein, “tablet” refers to compressed pharmaceutical dosage forms of all shapes and sizes, whether coated or uncoated.

The dosage forms of the present invention are produced by compacting or compressing a mixture or composition under pressure to form a stable three-dimensional shape (e.g., a tablet). In general, a compacted mixture has a density greater than that of the mixture prior to compaction. A compacted mixture can have almost any shape including concave and/or convex faces, rounded or angled corners, and a rounded to rectilinear shape. In some embodiments, the compressed dosage forms of the present invention comprise a rounded tablet having flat faces. The solid pharmaceutical dosage forms of the present invention can be prepared by any compaction and compression methods known by persons of ordinary skill in the art of forming compressed solid pharmaceutical dosage forms.

As used herein, a “particulate” refers to lansoprazole, excipients, and mixtures thereof that are composed of discrete particles.

In some embodiments, the lansoprazole and/or excipients in the solid pharmaceutical dosage forms are micronized. As used herein, “micronized” refers to particles of a composition that have been reduced to about 10 μm or less in diameter.

As used herein, the term “particle size” refers to particle diameter. Particle size and particle size distribution can be measured using, for example, a Hyac/Royco particle size analyzer, a Malvern particle size analyzer, a Beckman Coulter laser diffraction particle size analyzer, a Shimadzu laser diffraction particle size analyzer, or any other particle size measurement apparatus or technique known to persons of ordinary skill in the art. As used herein, the term “particle diameter” relates to a volumetric measurement based on an approximate spherical shape of a particle. The present invention can also comprise semi-spherical, ellipsoidal, or cylindrical particles without limitation. In addition to encompassing lansoprazole particles of a given size, the present invention is also directed to formulations and solid oral dosage forms wherein the distribution of particle sizes of lansoprazole, the lansoprazole spheroid, the bead portion, and excipients is controlled. As used herein, a “distribution” refers to the number or concentration (i.e., percentage) of particles having a certain size, or range of sizes, within a given lot, batch, or solid oral dosage form of the present invention.

As used herein, a “D₅₀” value refers to the particle size of a mixture, and specifically the diameter at which 50% (of the particles) of a composition or mixture have a larger equivalent diameter, and the other 50% of the particles have a smaller equivalent diameter. Thus, D₅₀ generally refers to the average particle diameter.

As used herein, “D₉₀” refers to the particle size of a mixture, and specifically the diameter at which about 90% of all measurable particles have a diameter equal to or less than the D₉₀ value, in microns, and about 10% of the measurable particles have a diameter greater than the D₉₀ value, in microns.

As used herein, “D₁₀” refers to the particle size of a mixture, and specifically the diameter at which about 10% of all measurable particles have a diameter equal to or less than the D₁₀ value, in microns, and about 90% of the measurable particles have a diameter greater than the D₉₀ value, in microns.

The distribution of particle sizes in a mixture can also be defined by the ratio D₁₀:D₅₀, the ratio D₁₀:D₉₀, and the ratio D₅₀:D₉₀.

The dosage forms of the present invention comprise one or more pharmaceutically acceptable excipients. As used herein, “pharmaceutically acceptable” refers to those excipients, compounds, materials, and/or compositions which are, within the scope of sound medical judgment, suitable for contact with the tissues of human beings and animals without excessive toxicity, irritation, allergic response, or other possible complications commensurate with a reasonable benefit/risk ratio. As used herein, the term “excipient” refers to the substances useful for combining with an active agent to provide a solid pharmaceutical dosage form suitable for administering to a subject in need thereof. In addition, one of skill in the art will recognize that pharmaceutically acceptable excipients can be used in the present invention including those listed in The Handbook of Pharmaceutical Excipients, 5th Ed., The Pharmaceutical Press and American Pharmacists Association, London, UK and Washington, DC (2006), which is incorporated herein by reference in its entirety.

The pharmaceutical compositions of the present invention comprise a lansoprazole spheroid. As used herein, a “lansoprazole spheroid” refers to a spherical, ellipsoidal, or semi-spherical moiety (e.g., a sphere) comprising lansoprazole and a pharmaceutically acceptable inert material and having an aspect ratio of about 0.75 to about 1.3. As used herein an “aspect ratio” refers to the ratio of the axes (i.e., x, y and z) that can define a spheroidal, ellipsoidal or semi-spherical moiety, wherein the aspect ratio can be any of the ratios x:y, x:z or y:z. For a sphere, the magnitudes of the spherical axes x, y and z are equal and the aspect ratio is 1. However, for semi-spherical and ellipsoidal moieties the aspect ratio is less than or greater than 1, wherein the magnitude of the deviation from 1 indicates the degree to which a moiety deviates in its shape from a sphere. In some embodiments, an aspect ratio can be determined by determining the axial parameters (i.e., x, y and z) for a lansoprazole spheroid or collection of spheroids (e.g., an individual measurement, an aggregate measurement, an average of measurements, and/or a distribution of measurements) and determining the ratios x:y, x:z and y:z.

In some embodiments, a lansoprazole spheroid of the present invention has an aspect ratio of about 0.75 to about 1.3, about 0.8 to about 1.25, about 0.85 to about 1.15, about 0.9 to about 1.1, about 0.95 to about 1.05, about 0.9, about 0.95, or about 1.

Not being bound by any particular theory, a lansoprazole spheroid having a spherical, semi-spherical or ellipsoidal shape with an aspect ratio of about 0.75 to about 1.3 results in a substantially homogeneous bead portion having a controllable particle size distribution, as well as enabling the formation and retention of a substantially homogeneous granulate-bead mixture.

In some embodiments, the lansoprazole spheroids have a mean diameter of about 275 μm to about 500 μm, about 300 μm to about 475 μm, about 325 μm to about 450 μm, about 350 μm to about 425 μm, about 380 μm, about 400 μm, or about 420 μm.

The lansoprazole and one or more pharmaceutically inert materials (i.e., excipients) can form a homogeneous distribution, a gradient, a multilaminate (i.e., a layered structure), or a combination thereof, within the lansoprazole spheroid. For example, in some embodiments, a lansoprazole spheroid comprises lansoprazole that has been mixed, granulated, or otherwise interspersed with a suitable binder to form substantially homogeneous spheroids. The spheroids can be compressed, ground, milled or otherwise mechanically shaped and screened to provide a collection of spheroids having a specified and substantially uniform particle size distribution.

In some embodiments, a lansoprazole spheroid comprises an inert core or seed having a lansoprazole coating layer thereon. Inert cores and seeds for use with the present invention can include an excipient chosen from: a sugar (e.g., NU-PAREIL® seeds, Chr. Hanson, Inc., Milwaukee, Wis.), a starch, a gelatinized starch, a glass (e.g., silica), a colloidal silica, a cellulose and/or derivatives thereof (e.g., CELLETS®, Pharmatrans Sanaq AG, Basel, Switzerland), sand, clay, and the like, and combinations thereof, as well as any other inert spheroidal or ellipsoidal materials known to persons of ordinary skill in the art. In some embodiments, cores or seeds suitable for use with the present invention are porous and/or have a roughened texture. In those embodiments in which a lansoprazole coating layer is applied to an inert core or seed, the lansoprazole can be present in the coating layer in a concentration of about 40% to about 70%, about 45% to about 65%, about 50% to about 60%, or about 55% by weight of the lansoprazole coating layer.

In some embodiments, a lansoprazole spheroid portion comprises a first portion of lansoprazole having thereon a second portion of lansoprazole present as a coating layer on the first portion. Both the first and second portions of the lansoprazole present in a lansoprazole spheroid can be mixed with one or more pharmaceutically inert excipients.

In some embodiments, a second active agent (in addition to lansoprazole) can form a coating layer on a lansoprazole spheroid, or a second spheroid portion comprising a second active agent in addition to lansoprazole can have a lansoprazole coating layer thereon.

In some embodiments, a portion of the lansoprazole spheroid further comprises an active agent in addition to lansoprazole. Active agents in addition to lansoprazole that are suitable for use with the present invention include, but are not limited to, a substituted benzimidazole (other than lansoprazole), an antacid, an analgesic, an anti-inflammatory, an antipyretic, an antibiotic, an antimicrobial agent, an anti-anorexic agent, an antihistamine, an anti-asthmatic agent, an anti-diuretic, an anti-migraine agent, a biological agent, an anti-spasmodic, a sedative, an anti-hyperactive agent, an anti-hypertensive agent, a tranquilizer, a decongestant, a beta-adrenergic antagonist, as well as any salts, esters, prodrugs, isomers and isomeric mixtures thereof, and combinations thereof.

The lansoprazole is mixed with one or more inert excipients to provide either of a lansoprazole coating layer and/or a core of a lansoprazole spheroid of the present invention. Inert excipients suitable for use in or on a spheroid include, but are not limited to, hydroxypropyl cellulose (“HPC” available as, e.g., KLUCEL® Type EF or KLUCEL® Type LF, Hercules Inc., Wilmington, Del.), hydroxyethyl cellulose (“HEC” available as, e.g., NATROSOL®, Hercules Inc., Wilmington, Del.), hydroxypropylmethyl cellulose, microcrystalline cellulose, and the like, and combinations thereof.

Not being bound by any particular theory, the spheroids of the present invention can be formulated to optimize one or more properties such as hardness, surface morphology (e.g., surface roughness and/or rugosity and the like), size distribution, and the like. In some embodiments, the lansoprazole spheroids comprise CELLETS® 200 microcrystalline cellulose spheres having a layer comprising lansoprazole in a binder of HPC thereon. HPC having a molecular weight of about 95,000 Daltons (“Da”) or less, about 80,000 Da or less, and combinations thereof is of particular use due to its ability to form a uniform coating having a hydrophilic surface capable of forming hydrogen bonds with a layer deposited thereon. Thus, the lansoprazole spheroids of the present invention can be prepared to provide a specified particle size distribution suitable for coating with additional layers. Additionally, the molecular weight of a binder present with lansoprazole in the lansoprazole spheroid can affect the release rate of the lansoprazole from the dosage forms of the present invention. Not being bound by any particular theory, the presence of a HPC having a molecular weight of about 95,000 Da or less or about 80,000 Da or less can ensure that lansoprazole present in a lansoprazole spheroid is rapidly released upon erosion of an enteric coating layer that surrounds the lansoprazole spheroid.

In some embodiments, a binder is present in a lansoprazole layer in a binder:lansoprazole ratio of about 1:1 to about 1:8 by weight, about 1:2 to about 1:6 by weight, or about 1:4 by weight.

In some embodiments, the lansoprazole spheroid has a basic pH of about 8 or more, about 9 or more, about 9.5 or more, about 10 or more, about 10.5 or more, about 8 to about 10, about 8.5 to about 10, or about 9 to about 10. Not being bound by any particular theory, a basic pH in the lansoprazole spheroid can stabilize the lansoprazole present in the dosage forms during manufacture of the dosage forms, storage, and administration (i.e., in the time period between administering a dosage form to a subject in need thereof and release of the lansoprazole from a dosage form).

The pH of the lansoprazole spheroid can be made basic via the addition of a stabilizer. In some embodiments, stabilizer can be present throughout a lansoprazole spheroid, or localized in a lansoprazole layer that coats an inert core or seed. In particular, a stabilizer can be present in a lansoprazole layer deposited on an inert seed or core, for example, using an aqueous dispersion comprising lansoprazole and the stabilizer. Suitable stabilizers include alkaline substances, addition salts that include an alkali metal and/or an alkaline earth metal, and the like. Stabilizers suitable for use with the present invention include, but are not limited to, carbonate salts (e.g., magnesium carbonate and the like), phosphate salts (e.g., aluminum phosphate and the like), citrate salts (e.g., magnesium citrate and the like), formate salts (e.g., calcium formate and the like), acetate salts (e.g., sodium acetate and the like), hydrates thereof, anhydrous forms thereof, and combinations thereof.

In some embodiments, a stabilizer is present in a stabilizer:lansoprazole ratio of about 1:1 to about 1:4 by weight, about 1:1 to about 1:3 by weight, or about 1:2 by weight. In some embodiments, a stabilizer is present in a stabilizer:lansoprazole ratio of about 1:1 to about 4:1 by mole, about 1:1 to about 3:1 by mole, or about 2:1 by mole.

Lansoprazole spheroids of the present invention are coated with one or more layers to form a bead (e.g., a bead portion). As used herein, a “bead” and a “bead portion” refers to a multilaminate structure comprising a lansoprazole spheroid having one or more coating layers disposed thereon. As used herein, a “final bead portion” refers to a lansoprazole spheroid having a deformable coating layer disposed thereon. Within the structure of the bead, the lansoprazole spheroids do not chemically react or interact with, or form a complex with, a layer applied thereto. For example, in some embodiments a lansoprazole spheroid and/or a core or seed present therein is substantially impermeable to, or cannot be substantially penetrated by, a coating layer applied thereto. Thus, in some embodiments, a lansoprazole spheroid comprises a seed or core having discrete layers thereon, wherein there is no substantial overlap between the layers.

As used herein, a “coating layer” refers to a composition that encompasses or otherwise surrounds a substrate to which the coating is applied. Substrates suitable for applying a coating layer thereto include lansoprazole spheroids (and cores or seeds present therein), beads, dosage forms (e.g., tablets), and the like. Generally, the coating layers of the present invention are of a substantially uniform thickness and composition. In some embodiments, a coating layer comprises discrete particles.

In some embodiments, the pharmaceutical compositions of the present invention further comprise a protective coating layer surrounding the lansoprazole spheroid. A protective coating layer can provide a flexible barrier that can prevent damage to the lansoprazole spheroid during subsequent coating processes, prevent interaction between components of an enteric coating layer and the lansoprazole present in a lansoprazole spheroid, and prevent interaction between other components or excipients present in the lansoprazole spheroid with an enteric coating layer. For example, an interaction between a stabilizer present in the lansoprazole spheroid can adversely affect the performance of an enteric polymer, thereby degrading the acid resistance of an enteric polymer, and possibly lowering the potency of the dosage forms.

Excipients suitable for use in a protective coating layer include, but are not limited to, a hydroxypropylmethyl cellulose (available as, e.g., METHOCEL®, Dow Chemical Co., Midland, Mich.), a hydroxypropyl cellulose (“HPC”), titanium dioxide, talc, and combinations thereof. In some embodiments, the protective coating layer comprises a HPC polymer having a molecular weight of about 95,000 Da or less. In some embodiments, a protective coating layer comprises a hydroxypropylmethyl cellulose having a methoxy content of about 28% or more and a HPC polymer having a molecular weight of about 95,000 Da or less.

Not being bound by any particular theory, the mechanical strength of a protective coating layer can be proportional to the molecular weight of a polymer present in the layer. Additionally, the presence of a binder such as titanium dioxide or talc can provide adhesion between a protective coating layer and the lansoprazole spheroid. Thus, the present invention provides a protective coating layer having an adhesive property and a controlled tensile strength and flexibility.

In some embodiments, a protective coating layer has a controlled pH of about 6.5 or more, about 7 or more, about 7.5 or more, or about 8 or more. Not being bound by any particular theory, a controlled pH of about 6.5 or more can ensure that a protective coating layer does not degrade any lansoprazole present on the surface of a lansoprazole spheroid. The pH of a protective coating layer can be controlled by the selection of excipients having a pH of about 6.5 or more, or by the addition of an appropriate amount of a stabilizer to the protective coating layer.

In some embodiments, a protective coating layer has a thickness of about 5 μm to about 50 μm, about 5 μm to about 25 μm, about 5 μm to about 15 μm, or about 10 μm. In some embodiments, a bead portion, after deposition of a protective coating layer onto a lansoprazole spheroid, has a D₅₀ of about 250 μm to about 500 μm, about 300 μm to about 480 μm, about 350 μm to about 460 μm, about 380 μm to about 430 μm, about 390 μm, or about 410 μm.

In some embodiments, a bead, after deposition of a protective coating layer onto a lansoprazole spheroid, has a D₁₀ of about 200 μm or greater, about 225 μm or greater, about 250 μm or greater, about 275 μm or greater, or about 300 μm or greater.

In some embodiments, a bead, after deposition of a protective coating layer onto a lansoprazole spheroid, has a D₉₀ of about 700 μm or less, about 650 μm or less, about 600 μm or less, or about 550 μm or less.

The bead portion of the present invention further comprises an enteric coating layer surrounding the lansoprazole spheroid. The enteric coating layer can be deposited directly on the lansoprazole spheroid, or the enteric coating layer can be deposited over an optional protective coating layer that has been applied to the lansoprazole spheroid. As used herein, an “enteric coating layer” refers to a layer comprising one or more enteric polymers. As used herein, an “enteric polymer” refers to a polymer having a solubility in an aqueous medium that is pH dependent (e.g., a polymer whose solubility depends on the acidity of an aqueous solution). Enteric polymers suitable for use with the present invention have a solubility that decreases as the pH decreases. Thus, there is a pH or range of pH values above which an enteric polymer for use with the present invention is soluble in an aqueous medium. Enteric polymers suitable for use with the present invention are insoluble in gastric juices, and can be formulated to dissolve in a region of the lower GI tract (e.g., any of the ileum, jejunum, cecum, colon or rectum). In some embodiments, enteric polymers suitable for use with the present invention dissolve in the ileum.

Not being bound by any particular theory, the pH below which an enteric polymer is insoluble in an aqueous medium can be controlled by the type of acidic side groups on the polymer, as well as the density of acidic side groups on the polymer. In the case of lansoprazole, an enteric layer protects the lansoprazole from the acidic environment of the stomach, permitting it to be released in the higher pH environment of the lower GI tract.

In some embodiments, an enteric polymer suitable for use with the present invention is insoluble at a pH of about 5 or less, about 5.5 or less, about 5.8 or less, about 6 or less, or about 6.5 or less. In some embodiments, an enteric polymer suitable for use with the present invention is a neutral polymer.

Enteric polymers suitable for use with the present invention include, but are not limited to, an anionic co-polymer of methacrylic acid and a (C₁-C₄)alkyl acrylate, a hydroxypropylmethyl cellulose acetate polymer, a neutral co-polymer of a (C₁-C₄)alkyl acrylate and a (C₁-C₄)alkyl methacrylate, a hydroxypropylmethyl cellulose phthalate polymer, a hydroxypropylmethyl cellulose succinate polymer, a cellulose acetate phthalate polymer, a polyvinylacetate phthalate polymer, and combinations thereof.

In some embodiments, the enteric polymers of the present invention can be chosen based on their ability to protect the lansoprazole spheroid at acidic pH and to form a flexible layer that can withstand compression of the granulate-bead mixture. In some embodiments, the enteric coating layer comprises an anionic enteric polymer having a molecular weight of about 250,000 Da or less in a concentration of about 50% to about 90%, about 55% to about 85%, about 60% to about 80%, about 65% to about 75%, or about 70% by weight of the enteric coating layer. In some embodiments, the enteric coating layer comprises a neutral enteric polymer having a molecular weight of about 800,000 Da or less in a concentration of about 10% to about 20%, or about 15% by weight of the enteric coating layer.

In some embodiments, the enteric coating layer comprises a first enteric polymer, which is an anionic co-polymer of methacrylic acid and ethylacrylate having a molecular weight of about 250,000 Da in a concentration of about 70% by weight, and a second enteric polymer which is a co-polymer of ethylacrylate and methyl methacrylate having a molecular weight of about 800,000 Da in a concentration of about 15% by weight of the enteric coating layer.

In some embodiments, the enteric coating layer further comprises a plasticizer suitable for controlling and enhancing the flexibility of an enteric coating, enhancing the coating performance during application, and the like. Plasticizers suitable for use with the present invention include, but are not limited to, acetyl tributyl citrate, dibutyl sebacate, dibutyl tartrate, dimethyl phthalate, diethyl phthalate, triethyl citrate, castor oil, triacetin, triacetin citrate, tributyl citrate, tripropionin, hydrogenated vegetable oil (e.g., LUBRITAB®, Penwest Pharmaceuticals Co., Patterson, N.J.), cetyl alcohol, cetylstearyl alcohol, fatty acids, glycerides and triglycerides (e.g., acetylated monoglyceride), glycerin, polyethylene glycol, polyoxyethylene glycols, propylene glycol, butyl lactate, ethyl glycolate, ethyl lactate, ethyl phthalylethyl glycolate, butyl phthalybutyl glycolate, sorbitol lactate, 1,2-butylene glycol, and the like, and combinations thereof.

In some embodiments, a plasticizer is present in one or more coating layers of the bead portion in a concentration of about 1% to about 20%, about 2% to about 20%, about 5% to about 20%, or about 10% to about 20% by weight of a coating layer.

In some embodiments, the enteric coating layer is present in a concentration of about 20% to about 60% by weight, about 25% to about 55% by weight, about 30% to about 50% by weight, about 35% to about 45% by weight, or about 40% by weight of the bead portion.

In some embodiments, the enteric coating layer has a thickness of about 80 μm to about 150 μm, about 90 μm to about 145 μm, about 100 μm to about 140 μm, about 110 μm to about 135 μm, about 120 μm to about 130 μm, or about 125 μm. In some embodiments, the bead portion, after deposition of an enteric coating layer onto a lansoprazole spheroid (optionally having a protective coating layer thereon), has a D₅₀ of 400 μm to about 700 μm, about 420 μm to about 650 μm, about 450 μm to about 600 μm, 400 μm to about 550 μm, about 420 μm to about 540 μm, about 450 μm, about 500 μm, or about 520 μm.

In some embodiments, the bead portion, after deposition of an enteric coating layer onto a lansoprazole spheroid (optionally having a protective coating layer thereon), has a D₁₀ of about 300 μm or greater, about 320 μm or greater, about 350 μm or greater, or about 380 μm or greater.

In some embodiments, the bead portion, after deposition of an enteric coating layer onto a lansoprazole spheroid (optionally having a protective coating layer thereon), has a D₉₀ of about 770 μm or less, about 740 μm or less, about 710 μm or less, or about 680 μm or less.

The solid pharmaceutical dosage forms of the present invention are acid resistant. As used herein, “acid resistance” refers to the percentage of lansoprazole that is released from the dosage forms after one hour of immersion in 475 mL of 0.1 N HCl in a USP Type II Paddle Apparatus at a paddle speed of 75 rpm. In some embodiments, the dosage forms of the present invention have an acid resistance of about 20% or less, about 15% or less, or about 10% or less. In some embodiments, the dosage forms of the present invention have an acid resistance of about 20% or less, about 15% or less, about 10% or less, about 5% or less, or about 3% or less. In some embodiments, the dosage forms of the present invention have an acid resistance of about 2% to about 20%, about 3% to about 15%, about 4% to about 10%, or about 5%.

In addition to acid resistance, the dosage forms of the present invention can also undergo rapid delivery of lansoprazole at pH of about 5.5 or greater. In some embodiments, the rate of lansoprazole release from the dosage forms can be quantified by immersing the dosage forms in 900 mL of phosphate buffer solution in a USP Type II Paddle Apparatus at a paddle speed of 75 rpm, under which conditions the release of lansoprazole from the dosage forms is about 75% or greater within 15 minutes, about 85% or greater within 20 minutes, and about 90% or greater within 30 minutes.

The solid pharmaceutical dosage forms of the present invention comprise a deformable coating layer surrounding the enteric layer. As used herein, a “deformable coating layer” refers to a layer that is plastically deformable and provides a cushioning effect during granulation and compression that prevents the enteric coating layer from becoming damaged or ruptured. For example, the deformable coating layer can significantly increase the crush strength of the beads of the present invention. As used herein, “crush strength” refers to the propensity of the beads to resist deformation upon application of an external pressure. Increasing crush strength correlates with increased resistance to deformation. Crush strength can be measured using, e.g., a texture analyzer apparatus. In some embodiments, the final bead portion has a crush strength of about 300 g or more, about 350 g or more, about 400 g or more, about 450 g or more, about 500 g or more, about 525 g or more, about 550 g or more, or about 600 g or more.

Additionally, the deformable coating layer is important for providing acceptable mouth feel for the orally disintegrating tablets. Specifically, the presence of a deformable coating layer diminishes gritty sensations that might otherwise be experienced by a subject upon disintegration of the dosage forms in the buccal cavity after administration.

Excipients suitable for use in the deformable coating layer include, but are not limited to, a polyvinyl alcohol, a polyether (e.g., polyethylene glycol and the like), a carboxyvinyl polymer (e.g., polyacrylic acid and the like), a cellulose derivative (e.g., hydroxypropyl methyl cellulose, sodium carboxymethyl cellulose, and the like), acacia, tragacanth, pectin, gelatin, derivatives thereof, and the like, and combinations thereof. In some embodiments, the deformable coating layer comprises a water-soluble wax such as, but not limited to, a polyethylene glycol. In some embodiments, the deformable coating layer comprises polyethylene glycol having a molecular weight of about 3,000 Da to about 8,000 Da, or about 3,000 Da to about 4,000 Da.

In some embodiments, the deformable coating layer comprises an excipient having a water solubility at 20° C. of about 0.01 mg/mL to about 10 mg/mL, about 0.05 mg/mL to about 5 mg/mL, or about 0.1 mg/mL to about 1 mg/mL.

In some embodiments, the deformable coating layer comprises a polymer having a glass transition temperature (“T_{g}”) of about −60° C. to about 30° C., about −50° C. to about 25° C., about −40° C. to about 20° C., or about −30° C. to about 15° C.

In some embodiments, the deformable coating layer comprises a polymer having a melting point of about 30° C. to about 150° C., about 40° C. to about 130° C., about 50° C. to about 110° C., about 55° C., about 75° C., or about 95° C.

In some embodiments, the deformable coating layer further comprises a binder such as, but not limited to, microcrystalline cellulose (available as, e.g. Avicel® PH-105, FMC Corporation, Philadelphia, Pa.), colloidal silicon dioxide (available as, e.g., CAB-O-SIL®, Cabot Corporation, Boston, Mass.), talc, and the like, and combinations thereof.

In some embodiments, the deformable coating layer is about 10% to about 30% by weight of the bead portion, about 10% to about 20% by weight of the bead portion, or about 15% to about 20% by weight of the bead portion.

In some embodiments, the final bead portion (i.e., the bead portion having a deformable coating layer thereon) of the present invention has a D₅₀ of 400 μm to about 700 μm, 425 μm to about 675 μm, about 450 μm to about 650 μm, about 475 μm to about 625 μm, about 500 μm to about 700 μm, about 525 μm to about 675 μm, about 500 μm to about 600 μm, about 450 μm to about 600 μm, about 500 μm, about 530 μm, or about 560 μm.

In some embodiments, the final bead portion has a D₁₀ of about 350 μm or greater, about 375 μm or greater, about 400 μm or greater, or about 425 μm or greater.

In some embodiments, the final bead portion has a D₉₀ of about 800 μm or less, about 775 μm or less, about 750 μm or less, about 725 μm or less, or about 700 μm or less.

In some embodiments, the final bead portion has a D₁₀:D₅₀ ratio of about 1:1.5, about 1:1.4, about 1:1.3, or about 1:1.2. In some embodiments, the final bead portion has a D₅₀:D₉₀ ratio of about 1:1.6, about 1:1.5, about 1:1.4, or about 1:1.3.

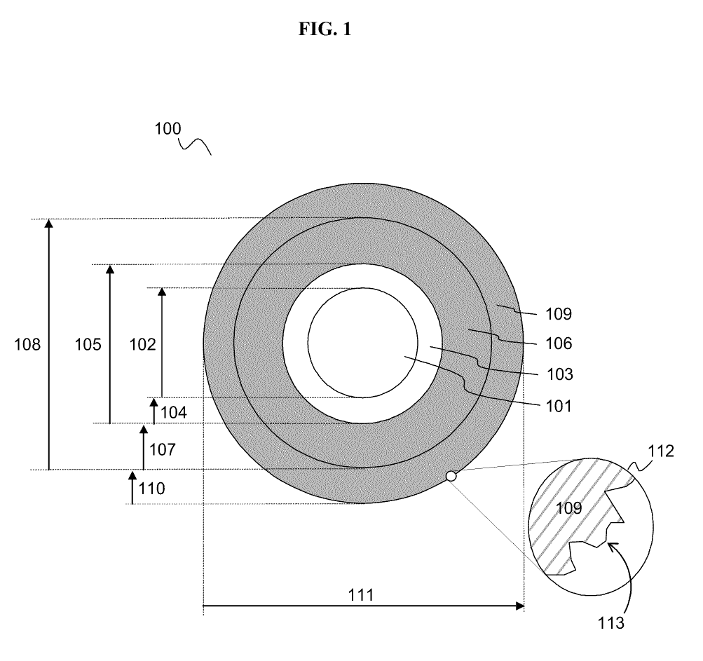

Thus, in some embodiments, the bead portion comprises a lansoprazole spheroid having a protective coating layer thereon, and an enteric coating layer and a deformable coating layer surrounding the protective coating. FIG. 1 provides a cross-sectional schematic representation of an exemplary embodiment of a bead portion, 100, of the present invention. Referring to FIG. 1, the bead portion, 100, comprises a lansoprazole spheroid, 101, containing lansoprazole as either: a) homogeneously distributed throughout the spheroid; b) as a lansoprazole coating layer on an inert core or seed; or c) a combination thereof. The lansoprazole spheroid has a diameter indicated by the magnitude of vector 102. In some embodiments, the bead portion, 100, further comprises an optional protective coating layer, 103, which can surround the lansoprazole spheroid, the optional protective coating layer having a thickness indicated by the magnitude of vector 104. After deposition of a protective coating layer, 103, the bead portion has a diameter indicated by the magnitude of vector 105. Surrounding the optional protective coating layer, 103, is an enteric coating layer, 106, having a thickness indicated by the magnitude of vector 107. After deposition of the enteric coating layer, 106, the bead portion has a diameter indicated by the magnitude of vector 108. The bead portion, 100, further comprises a deformable coating layer, 109, which surrounds the enteric coating layer, 106; the deformable coating layer, 109, having a thickness indicated by the magnitude of vector 110. After deposition of the deformable coating layer, 109, the bead portion, 100, has a diameter indicated by the magnitude of vector 111. In some embodiments, the deformable coating layer has a roughened outer surface. Referring to inset, 112, a schematic representation of a roughened surface, 113, of the deformable coating layer, 109, is provided.

As used herein, “surface roughness” refers to an arithmetic average of multiple two-dimensional roughness profiles measured across the surface of a bead of the present invention, wherein the roughness is the absolute value of the vertical deviations of a real surface from its ideal form. In some embodiments, a deformable coating layer has a surface roughness of about 50 nm to about 10 μm, about 100 nm to about 5 μm, about 500 nm to about 2 μm, about 700 nm, about 850 nm, or about 1 μm.

As used herein, “rugosity” refers to the actual surface area of a bead of the present invention divided by the geometric surface area of the bead. In some embodiments, a deformable coating layer has a rugosity of about 1.05 to about 1.2, about 1.05 to about 1.15, or about 1.05 to about 1.1.

Surface roughness and/or rugosity can be measured using various contact (e.g., profilometry and the like) and non-contact methods (e.g., interferometry, confocal microscopy, and the like) known to persons of ordinary skill in the art.

Not being bound by any particular theory, a roughened surface can prevent segregation of beads from the final blend during manufacturing (i.e., granulation and compression) of the dosage form.

The orally disintegrating solid pharmaceutical dosage forms of the present invention comprise a compacted granulate-bead mixture. In some embodiments, the bead portion and granulate portion are present in a bead:granulate ratio of about 3:1 to about 1:3, about 2:1 to about 1:2, about 3:2 to about 2:3, about 5:4 to about 4:5, or about 1:1, by weight.

The bead portion is combined with a granulate portion to form a final blend that is compressed to provide the solid pharmaceutical dosage forms of the present invention. The compressed granulate-bead mixture must exhibit rapid disintegration and good mouth feel while being of suitable mechanical strength to prevent rupture of the bead portion during compression or disintegration. Thus, as used herein, a “granulate portion” refers to a dry mixture that when compressed, exhibits orally disintegrating properties. As used herein, a “granulate-bead mixture” refers to a solid dispersion, suspension, solution, and the like comprising a granulate portion and a bead portion.

The granulate portion of the present invention comprises a sugar alcohol having a solubility of about 150 mg/mL or more in saliva and/or water, and an excipient that undergoes a volume expansion of about 75% or more upon contact with saliva.

Not being bound by any particular theory, a water-soluble excipient capable of undergoing rapid dissolution in saliva can provide a fluidic or viscous sensation in the mouth that provides a positive or “good” mouth feel, while a super-disintegrant capable of wicking saliva into the solid pharmaceutical dosage form and undergoing rapid volume expansion can contribute to rapid disintegration of the granulate portion of the solid pharmaceutical dosage forms. However, the balance of these properties is crucial for providing a solid pharmaceutical dosage form that undergoes rapid disintegration and also provides acceptable organoleptic properties such as mouth feel and taste.

As used herein, solubility (in water, saliva, or any other fluid) is determined at 20° C. Sugar alcohols suitable for use with the present invention include, but are not limited to, mannitol, arabinose, dextrose, erythritol, galactose, inositol, lactitol, maltitol, maltose, pentose, sorbitol, sucrose, tagatose, trehalose, xylitol, xylose, and combinations thereof. In some embodiments, the sugar alcohol comprises mannitol. Directly compressible mannitol (e.g., spray-dried mannitol) is particularly useful as an excipient in the granulate portion because it can be used to prepare compressed dosage forms having a low hardness using standard tableting equipment. Thus, the dosage forms of the present invention can be easily manufactured without requiring special tableting apparatus, punches, or dies.

In some embodiments, the granulate portion comprises a sugar alcohol in a concentration of about 1% to about 20% by weight, about 3% to about 15% by weight, or about 5% to about 10% by weight. In some embodiments, the solid pharmaceutical dosage forms comprise a sugar alcohol in a concentration of about 10% or less, about 5% or less, about 1% to about 10%, about 2% to about 5%, about 3%, or about 4% by weight of the solid pharmaceutical dosage forms.

Excipients that undergo a volume expansion of about 75% or more upon contact with saliva are generally disintegrants known as “super-disintegrants” such as, but not limited to, crospovidone, cross-linked polymers of carboxymethylcellulose sodium (e.g., croscarmellose sodium, available as SOLUTAB®, Blanver Farmoquimica, Ltda., Cotia, Brazil; Ac-DI-SOL®, FMC Corp., Philadelphia, Pa.; or VIVASOL®, J. Rettenmaier & Sohne GmbH+Co. KG Ltd., Rosenberg, Germany), cross-linked derivatives of starch (e.g., sodium starch glycolate, available as PRIMOJEL®, Campina Nederland Holding B. V., Zaltbommel, Netherland Antilles; and EXPLOTAB®, Edward Mendell Co., Inc., Carmel, N.Y.), pregelatinized starch, and combinations thereof.

A disintegrant that can be used with the dosage forms of the present invention is crospovidone, which is a cross-linked homopolymer of N-vinylpyrrolidone (available as, e.g., POLYPLASDONE XL® and POLYPLASDONE XL-10®, International Specialty Products (ISP), Wayne, N.J.; see e.g., Pharmaceutical Technical Bulletin for POLYPLASDONE XL-10®, ISP).

Disintegrants are typically present in directly compressed tablet formulations (e.g., directly compressed orally disintegrating tablets) in a concentration of about 2% to about 8% by weight of the tablet. Disintegrants are not typically present in orally disintegrating dosage forms in a concentration greater than about 20% by weight. This is because it is commonly held that a super-disintegrant in concentrations of greater than 20% by weight can cause deleterious effects such as reduced mechanical stability, poor mouth feel, and increased friability.

On the other hand, in some embodiments, a disintegrant is present in the granulate portion in a concentration of about 20% or more, about 25% or more, about 30% or more, about 35% or more, about 40% or more, about 45% or more, or about 50% or more by weight. In some embodiments, the granulate portion comprises a super-disintegrant in a concentration of about 20% to about 60%, about 25% to about 55%, about 30% to about 55%, or about 35% to about 50% by weight.

In some embodiments, a granulate portion further comprises a hydrophilic, water-insoluble excipient suitable for acting as a wicking agent, diluent, binder, and the like. In some embodiments, a diluent and/or a binder can facilitate at least one of compression and/or disintegration of the dosage forms of the present invention. Not being bound by any particular theory, compression decreases the free volume that is normally associated with a mixture, and results in a composition having an increased density and decreased free volume, which can deleteriously affect the disintegration rate. Thus, a diluent and/or binder having a high porosity and/or free volume can help to ensure that the dosage forms of the present invention are efficiently penetrated by water or saliva and undergo rapid disintegration. Free volume and/or porosity can relate to the density of an excipient, and binders and/or diluents suitable for use with the present invention can therefore be selected based upon their density. In some embodiments, diluents and binders suitable for use with the present invention have a density of about 0.7 g/cm³ or less, of about 0.6 g/cm³ or less, or of about 0.5 g/cm³ or less. Hydrophilic, water insoluble excipients suitable for use with the present invention include, but are not limited to, microcrystalline cellulose, a phosphate (e.g., calcium phosphate), a sulfate (calcium sulfate), a carbonate (e.g., calcium carbonate), a silicate (e.g., aluminum magnesium silicate, aluminum magnesium metasilicate, aluminum silicate, bentonite, silica gel), a hydrotalcite, a metal hydroxide (e.g., aluminum hydroxide), a metal oxide (e.g., titanium dioxide), and the like, and combinations thereof.

In some embodiments, a hydrophilic, water-insoluble excipient is present in a concentration of about 10% to about 40%, about 15% to about 35%, about 20% to about 40%, or about 20% to about 35% by weight of the granulate portion.

In some embodiments, the dosage forms comprise microcrystalline cellulose in a concentration of about 10% to about 40%, about 15% to about 35%, about 20% to about 40%, or about 20% to about 35% by weight of the granulate portion.

In some embodiments, the orally disintegrating solid pharmaceutical dosage forms of the present invention further comprise a viscosity-imparting agent suitable for thickening an aqueous solution. Viscosity-imparting agents suitable for use with the present invention include thixotropic agents (i.e., an agent that enhances the stability of a solution, dispersion, or mixture upon standing). Viscosity-imparting agents suitable for use with the present invention include, but are not limited to, hydroxypropyl methylcellulose, hydroxyethyl cellulose, hydroxypropyl cellulose, and combinations thereof.

In some embodiments, the orally disintegrating solid pharmaceutical dosage forms of the present invention further comprise a lubricant that can prevent adhesion of the granulate-bead mixture to a surface (e.g., a surface of a mixing bowl, a compression die and/or punch). A lubricant can also reduce interparticle friction within the granulate and aid in the ejection of a compressed dosage form from a die cavity after compression. Lubricants suitable for use with the present invention include, but are not limited to, magnesium stearate, calcium stearate, zinc stearate, sodium stearate, stearic acid, aluminum stearate, leucine, glyceryl behenate, sodium lauryl sulfate, sodium stearyl fumarate (e.g., PRUV®, Sohne GmbH & Co., Rosenberg, Germany), hydrogenated vegetable oil, and combinations thereof. In some embodiments, the lubricant comprises magnesium stearate, sodium stearyl fumarate, or a combination thereof.

In some embodiments, a lubricant is present in a concentration of about 10% or less, about 8% or less, about 5% or less, about 2% or less, about 0.1% to about 10%, about 0.1% to about 6%, about 0.1% to about 5%, about 0.1% to about 4%, about 0.1% to about 3%, or about 0.1% to about 2% by weight of the granulate portion.

In some embodiments, the orally disintegrating solid pharmaceutical dosage forms of the present invention further comprise a glidant that can improve the flow characteristics of the granulate and/or the granulate-bead mixture. Non-limiting examples of glidants suitable for use with the present invention include colloidal silicon dioxide (e.g., CAB-O-SIL®, Cabot Corp., Boston, Mass.; or AEROSIL®, Degussa AG, Frankfurt, Germany), talc, and the like, and combinations thereof. In some embodiments, a glidant is present in a concentration of about 10% or less, about 5% or less, about 0.1% to about 10%, or about 0.2% to about 5% by weight of the granulate portion.

In some embodiments, the orally disintegrating solid pharmaceutical dosage forms of the present invention further comprise a flavorant suitable for providing a pleasant taste to a subject upon administration of the dosage forms to a subject in need thereof. Flavorants can be combined, as desired, to produce a particular flavor mixture which is compatible with a particular medication. Flavorants suitable for use with the present invention include, but are not limited to, strawberry, raspberry, cherry, almond, citrus fruit, vanilla, vanilla cream, mint, spearmint, wintergreen, grape, coconut, chocolate, menthol, licorice, butterscotch and combinations thereof. Citrus fruit flavorings suitable for use with the present invention include, but are not limited to, orange, tangerine, lemon, lime, lemon-lime and combinations thereof.

A flavorant can be present in the granulate portion, one or more coating layers of the bead portion, or as a coating layer on the solid pharmaceutical dosage forms in a concentration of about 30% or less, about 20% or less, about 10% or less, about 5% or less, about 0.05% to about 30%, about 0.1% to about 20%, or about 0.5% to about 10% by weight of the dosage forms.

In some embodiments, the orally disintegrating solid pharmaceutical dosage forms of the present invention further comprise a flavor enhancer. A “flavor enhancer” refers to an excipient that can be added to the dosage forms to achieve a better tasting product or provide a more pleasant mouth feel during administration. Non-limiting examples of flavor enhancers suitable for use with the present invention include ribotide and monosodium glutamate.

In some embodiments, the orally disintegrating solid pharmaceutical dosage forms of the present invention further comprise a natural or artificial sweetener to improve the taste of the dosage forms. A “sweetener” refers to an excipient that imparts a sweet taste to the dosage forms. Sweeteners suitable for use with the present invention can comprise an excipient having a hydrophilic, water-soluble excipient having a —CHOH functional group, a sugar-free sweetener, a natural sweetener, an artificial sweetener, and combinations thereof. In some embodiments, the orally disintegrating solid pharmaceutical dosage forms of the present invention are substantially free of sugar (i.e., “sugar-free”). “Sugar-free” can also refer to a dosage form that is substantially free of complex carbohydrates and/or polysaccharides that can be readily converted to sugars in the oral cavity. A sugar-free dosage form can offer reduced caloric value, reduced dental caries and other dental hygienic issues, and can be preferable for administering to subjects seeking to control sugar intake (i.e., diabetic subjects).

Sugar-free sweeteners suitable for use with the present invention include, but are not limited to, aspartame, saccharin and salts thereof (e.g., saccharin sodium), acesulfame potassium, altitame, cyclamic acid and its salts (e.g., sodium cyclamate), dihydrochalcones, glycerrhizinate, monellin, neotame, saccharin, stevioside, sucralose, thaumatin, and combinations thereof. In some embodiments, a sweetener for use with the present invention comprises sucralose, aspartame, or a combination thereof. A sweetener can be present in the granulate portion, a coating layer of the bead portion, or in a coating layer on the dosage forms of the present invention in a concentration of about 0.005% to about 8%, about 0.01% to about 7%, about 0.01% to about 6%, or about 0.1% to about 5% by weight of the dosage forms. In some embodiments, the orally disintegrating solid pharmaceutical dosage forms of the present invention comprise aspartame in a concentration of about 2% to about 6% by weight of the granulate portion or dosage forms.

In some embodiments, the orally disintegrating solid pharmaceutical dosage forms of the present invention further comprise a colorant. A “colorant” refers to a substance that can be added to the dosage forms in the deformable coating layer, the granulate and/or as a coating on the dosage forms to enhance or modify their color or appearance. A colorant can also be added to the pharmaceutical dosage forms as a code or identifier (i.e., to indicate the manufacturer or dosage). Any type of colorant (i.e., “natural color” and/or “artificial color” such as F.D.&C. dyes) known to be “generally regarded as safe” by the FDA, and thus generally used in the confectionary trade, or otherwise approved by the FDA for use in pharmaceutical preparations, can be used with the present invention. In some embodiments, a colorant is iron oxide.

In some embodiments, the orally disintegrating solid pharmaceutical dosage forms of the present invention have a weight of about 200 mg to about 1000 mg, about 250 mg to about 800 mg, about 275 mg to about 700 mg, or about 300 mg to about 600 mg. As the amount of lansoprazole present in the orally disintegrating solid pharmaceutical dosage forms of the present invention is varied, the weight of the dosage forms can increase or decrease in a proportional manner.

The orally disintegrating solid pharmaceutical dosage forms of the present invention undergo complete disintegration without the use of effervescent agents. Suitable methods for determining the disintegration time and rate of disintegration include the use of an automated disintegrating tester (e.g., available from Erweka America Corp., Annandale, N.J.) or a texture analyzer (e.g., available from Texture Technologies Corp., Scarsdale, N.Y.), and using methods described in, for example, El-Arini, S. K. and Clas S. D., “Evaluation of disintegration testing of different fast dissolving tablets using the texture analyzer,” Pharm. Dev. Technol. 7:361-371 (2002), which is incorporated herein by reference in its entirety.

Typically, the volume of saliva in the buccal cavity of a human subject is about 2 mL. Not being bound by any particular theory, this volume of liquid acts as a medium into which the orally disintegrating dosage forms of the present invention disintegrate. During disintegration, hydrophilic water-insoluble excipients fragment into smaller particles, while hydrophilic water-soluble excipients dissolve in the saliva present in the buccal cavity. Thus, the orally disintegrating solid pharmaceutical dosage forms of the present invention comprise a hydrophilic excipient, i.e., an excipient having an affinity for aqueous liquids. Hydrophilic excipients suitable for use with the present invention include both hydrophilic excipients that are soluble in water, and those that are insoluble in water. In some embodiments, the dosage forms of the present invention comprise a saliva-generating excipient suitable for increasing the amount of saliva in the buccal cavity upon administration to a subject.

In some embodiments, the orally disintegrating solid pharmaceutical dosage forms of the present invention disintegrate without effervescence in about 2 mL or less of water or saliva in about 60 seconds or less, about 30 seconds or less, about 20 seconds or less, about 10 seconds or less, or about 8 seconds or less. In some embodiments, the orally disintegrating solid pharmaceutical dosage forms of the present invention disintegrate without effervescence upon administration to a subject in need thereof without administering water to the subject, in about 60 seconds or less, about 45 seconds or less, about 30 seconds or less, about 15 seconds or less, about 10 seconds or less, or about 8 seconds or less.

The orally disintegrating solid pharmaceutical dosage forms of the present invention also have excellent “mouth feel.” As used herein, “mouth feel” refers to the presence of grit or debris in the buccal cavity after disintegration of a non-effervescent, orally disintegrating solid pharmaceutical dosage form. Mouth feel relates to the bulkiness of the remaining tablet mass after disintegration, and can be an important parameter for maintaining patient compliance. Not being bound by any particular theory, excipients having appreciable solubility in saliva will largely dissolve during disintegration, whereas low-solubility excipients such as super-disintegrants will frequently break apart but not dissolve. Thus, the presence of a high concentration of super-disintegrant and/or other excipients that are largely insoluble in saliva in the solid pharmaceutical dosage forms can have a significantly negative effect on mouth feel. For example, the bead portion of the solid dosage forms remains largely intact upon administration and disintegration of the dosage forms, which can contribute significantly to a gritty sensation in the buccal cavity of a subject. It has been recognized that orally disintegrating dosage forms that include materials and/or beads having a particle size on the order of several hundred microns or larger can present a gritty texture and poor taste upon administration.

Suitable methods for determining mouth feel include measuring with a texture analyzer, blinded screening using placebo formulations, and the like, and combinations thereof. Using a texture analyzer, mouth feel is measured as the difference (Δ) between the thickness (h) of a dosage form and the penetration distance (d) of a liquid medium (e.g., synthetic saliva) into the dosage form during the course of a pre-determined time interval (e.g., one minute). Mouth feel improves as the value A is minimized (i.e., as the percentage amount of the solid pharmaceutical dosage form is dissolved in the liquid medium, the mouth feel increases). In some embodiments, mouth feel can also be quantified using blinded placebo trials, in which volunteer human subjects are administered placebo orally disintegrating dosage forms and the subjects rate the pleasantness of the mouth feel of the dosage forms on a scale of 1-5, wherein “1” refers to a gritty, unpleasant taste and/or texture and “5” refers to a pleasing, well-liked taste and/or texture.

Generally, it is assumed that to maintain an acceptable mouth feel, the concentration of water-insoluble excipients should be minimized, and in particular the concentration of a super-disintegrant should be maintained at about 20% or less by weight of a non-effervescent, orally disintegrating dosage form. This is because disintegrants and super-disintegrants are not typically soluble in saliva or water, and therefore form gritty particles upon disintegration. Thus, it is commonly held that minimizing the concentration of a disintegrant can improve the mouth feel of a non-effervescent, orally disintegrating dosage form. Surprisingly, the present invention has found that a non-effervescent, orally disintegrating solid pharmaceutical dosage form can be prepared which retains a superior mouth feel, while containing a higher concentration of disintegrant compared to what is typically used in previous orally disintegrating dosage forms. Moreover, the solid pharmaceutical dosage forms of the present invention contain relatively low concentrations of excipients having appreciable water-solubility such as sugar alcohols. Instead the deformable coating layer on the bead portion significantly enhances the mouth feel properties of the dosage forms upon administration to a subject. In some embodiments, upon administration to a subject the deformable coating layer undergoes partial dissolution in the buccal cavity to provide a viscous medium that surrounds the bead portion. Not being bound by any particular theory, partial dissolution of the deformable coating layer upon administration of the dosage forms can mask the particulate nature of the bead portion, thereby significantly enhancing the mouth feel of the dosage forms.

Not being bound by any particular theory, mouth feel can also be controlled and optimized by controlling the particle size of dry granulate excipients, and in particular the particle size of water insoluble dry granulate excipients. In some embodiments, the dry granulate has a D₅₀ of about 150 μm or less, about 120 μm or less, about 110 μm or less, about 100 μm or less, or about 80 μm or less. In some embodiments, the dry granulate has a D₅₀ of about 30 μm to about 150 μm, about 50 μm to about 120 μm, about 70 μm to about 110 μm, about 90 μm, or about 105 μm.

In some embodiments, the concentration of the super-disintegrant and sugar alcohol can be selected to optimize the physical integrity of the dosage forms of the present invention (e.g., to minimize friability).

The solid pharmaceutical dosage forms of the present invention have a hardness and friability which makes them stable during preparation, packaging and storage. As used herein, “hardness” refers to the degree of force required to break, crumble or crack the pharmaceutical solid pharmaceutical dosage form of the present invention. Hardness can be described in units of kilograms/mm² (“kg/mm²”), pounds/in² (“psi”), Pascals (“Pa”), Newtons/m² (“N/m²”), kiloponds (“kp”), mohls or arbitrary units. The hardness of the orally disintegrating solid pharmaceutical dosage forms can be measured using a tablet hardness tester, or by any other suitable method for determining hardness that is known to a person of ordinary skill in the art.

In some embodiments, the orally disintegrating solid pharmaceutical dosage forms of the present invention have a hardness of about 5 kp or less. Not being bound by any particular theory, a hardness of about 5 kp or less can enhance penetration of liquid into the dosage forms and facilitate disintegration. When hardness exceeds 5 kp, compressed dosage forms can exhibit unsatisfactory disintegration and can degrade manufacturing equipment. However, if hardness is less than 1 kp, compressed dosage forms can exhibit unacceptable friability. Thus, in some embodiments the solid pharmaceutical dosage forms have a hardness of about 1 kp to about 5 kp, about 1 kp to about 4 kp, about 1.5 kp to about 4 kp, about 2 kp to about 5 kp, about 2 kp to about 4 kp, about 2 kp to about 3 kp, about 2 kp, about 2.5 kp, or about 3 kp.

As used herein, “friability” refers to the tendency of a solid pharmaceutical dosage form to crumble. Friability can be measured by allowing the dosage forms to roll and fall within a rotating apparatus known as a friabilator, or by any other suitable method for determining hardness that is known to a person of ordinary skill in the art. In some embodiments, the dosage forms have a friability of about 10% or less, about 5% or less, about 3% or less, or about 1% or less.

The orally disintegrating solid pharmaceutical dosage forms of the present invention are delayed-release dosage forms. As used herein, “delayed-release” refers to the controlled release of an active pharmaceutical ingredient from a dosage form wherein the lansoprazole is released about 0.8 hour or more, about 1 hour or more, about 1.2 hours or more, about 1.5 hours or more, about 1 hour to about 2 hours, or about 1 hour to about 1.5 hours after oral administration of the dosage form to a subject in need thereof. In some embodiments, a delayed release of lansoprazole from the dosage forms of the present invention can be controlled by one or more of the solubility and permeability properties of the enteric coating layer, the permeability properties of the lansoprazole spheroid, and combinations thereof.

As used herein, “dissolution” refers to the process by which lansoprazole dissolves into solution from the pharmaceutical dosage forms of the present invention. The dissolution rate of lansoprazole can be measured using, for example, a USP Type I or Type II Dissolution Apparatus. In some embodiments, the dissolution of lansoprazole from the solid pharmaceutical dosage forms is measured using a USP Type II Paddle Apparatus containing 475 mL of 0.1 N HCl at a paddle speed of 75 rpm, followed by a USP Type II Paddle Apparatus containing 900 mL phosphate buffer at pH 6.8 at a paddle speed of 75 rpm.

### Processes to Prepare the Pharmaceutical Dosage Forms

The present invention is also directed to a process to prepare a non-effervescent, orally disintegrating solid pharmaceutical dosage form, the process comprising:

mixing a bead portion comprising lansoprazole spheroids having an enteric coating layer and a deformable coating layer thereon, wherein the deformable coating layer is about 10% to about 30% by weight of the bead portion and the bead portion has a D₅₀ of 400 μm or greater with a granulate portion comprising a disintegrant in a concentration of about 20% to about 60% by weight of the granulate, wherein the bead portion and the granulate portion are present in a ratio of about 1:3 to about 3:1, to form a substantially homogeneous granulate-bead mixture; and
compacting the granulate-bead mixture at a pressure of about 50 kN or less to provide the non-effervescent, orally disintegrating solid pharmaceutical dosage form, wherein upon oral administration without water the solid pharmaceutical dosage form disintegrates in about 60 seconds or less.

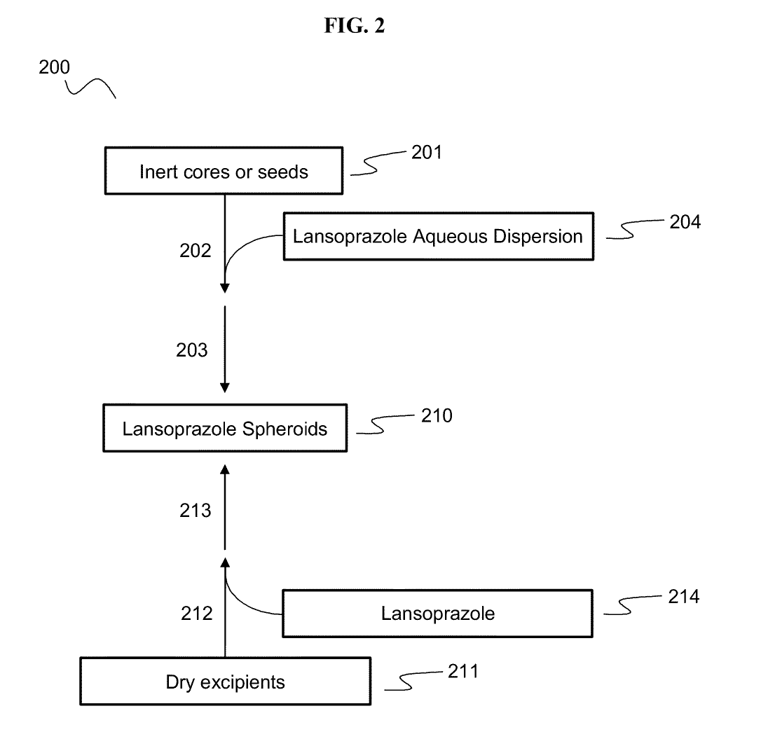

The granulate-bead mixture is prepared by mixing a granulate portion with a bead portion. The bead portion is prepared by first providing lansoprazole spheroids. FIG. 2 provides a flow chart representing processes for preparing lansoprazole spheroids of the present invention. Referring to FIG. 2, in an embodiment, inert cores or seeds, 201, are coated, 202, with a lansoprazole aqueous dispersion, 204, to provide a lansoprazole spheroid that is sized, 203, to provide lansoprazole spheroids of a specified particle size, 210. In an embodiment, dry excipients, 211, are combined with lansoprazole, 214, which can be wet (e.g., an aqueous dispersion) or dry, and then mixed with one or more optional diluents, binders and the like. The lansoprazole-dry excipient mixture can be compressed, spheronized, or otherwise processed, 212, and then sized, 213, to provide lansoprazole spheroids, 210, having a uniform and specified particle size distribution.

The lansoprazole spheroids are then sequentially coated with an optional protective coating layer, an enteric coating layer, and a deformable coating layer. The process can comprise one or more of drying, curing, and/or sizing between application of the various coating layers to ensure that a stable bead portion having a specified and substantially uniform size distribution of 400 μm or greater is provided.

In some embodiments, the coating layers are applied from an aqueous suspension or solution that minimizes the use of organic solvents. The coating layers can be applied, e.g., in a fluid bed apparatus. The coating processes of the present invention can be performed at the same or different temperatures in a range of about 20° C. to about 90° C.

The granulate portion can be prepared using one or more wet and/or dry granulation and/or milling processes. In some embodiments, a mixture is milled prior to or after wet granulation. In some embodiments, a wet granulate is milled directly, or alternatively dried, and then milled to prepare a mixture having a controlled, reduced particle size and a uniform particle size distribution. In some embodiments, the granulate portion has a D₅₀ of about 400 μm or less, about 350 μm or less, about 300 μm or less, about 250 μm or less, about 200 μm or less, about 150 μm or less, about 100 μm or less, about 50 μm to about 400 μm, about 50 μm to about 350 μm, about 50 μm to about 300 μm, about 50 μm to about 250 μm, or about 50 μm to about 200 μm.

In some embodiments, a first granulate portion is prepared separately from the bead portion by adding an aqueous solution comprising a sugar alcohol to a dry mixture comprising a super-disintegrant and a hydrophilic, water-insoluble excipient to form a wet granulate. Additional excipients such as a sweetener, a glidant, and the like can be present in the wet granulate. The wet granulate is optionally dried and then milled, followed by drying to ensure a water content of about 3% or less by weight. This dry granulate can undergo additional milling, screening, or other sizing processes to provide the first granulate, which has a substantially uniform composition, and specified flow and size.

A first granulate can alternatively be prepared by a dry granulation process, a slugging process, a roller-compaction process, or another process known to a person of ordinary skill in the art.

Not being bound by any particular theory, the presence of a disintegrant during wet granulation can ensure efficient mixing of the excipients during wet granulation. Because wet granulation involves spraying a solution onto a dry mixture, deviations in the process can result in the formation of a paste or dough that is unsuitable for further processing. The presence of a super-disintegrant and a hydrophilic, water-insoluble excipient can minimize process deviations during a wet granulation. The presence of a super-disintegrant in the wet-granulation process can also ensure that a super-disintegrant is mixed homogeneously throughout the granulate.

In some embodiments, a first granulate is combined with the bead portion to prepare a free-flowing and cohesive mixture. An additional amount of a super-disintegrant is then added to the mixture, along with optional excipients such as, but not limited to, a flavorant, a sweetener, a glidant, and the like, to provide the granulate-bead mixture. Because compressed dosage forms are also susceptible to problems such as, for example, particle fusion during and after compression, incorporating a super-disintegrant in multiple mixing operations can ensure that super-disintegrant particles having a range of particle sizes are incorporated homogeneously throughout the granulate, thereby minimizing the formation of a gritty residue or particles in the mouth of a subject due to incomplete disintegration of the dosage forms.

A lubricant is typically added to the granulate-bead mixture immediately prior to compression, followed by additional mixing to provide a substantially homogeneous composition that can be compressed to form a non-effervescent, orally disintegrating solid pharmaceutical dosage form of the present invention using, for example, a tablet press.

For facile mixing and loading of the granulate-bead mixture into tablet punches, the granulate-bead mixture has excellent flow characteristics and uniformity. As used herein, a “flow index” refers to a flow characteristic of a solid or a solid solution, as described in ASTM flow standard (Committee D18.25, D6682-01, D6128-97). In some embodiments, a flow index can be determined relative to a standard such as BCR116 standard limestone powder. In some embodiments, the granulate portion has a flow index of about 50 or more, about 60 or more, about 70 or more, about 80 or more, about 90 or more, or about 100 or more.

The process of the present invention can be run continuously without a requirement for any special environmental control (e.g., temperature, humidity, oxygen concentration, etc.). In particular, the process of the present invention avoids lyophilization and the costs associated with it, as well as processes that modify any crystalline properties of lansoprazole. Additionally, the process of the present invention can be performed using a conventional tableting apparatus, which provides for good scalability and reproducibility. Thus, the process of the present invention can be used to mass-produce low-cost pharmaceutical dosage forms which are stable under humid conditions and which disintegrate without effervescence.

The directly compressed non-effervescent, orally disintegrating solid pharmaceutical dosage forms have a hardness of about 1 kp to about 5 kp, which is sufficient to permit penetration of saliva into the dosage forms while maintaining the structural integrity of the dosage forms during storage and shipment. The compressed orally disintegrating solid pharmaceutical dosage forms of the present invention are typically compressed under a pressure of about 50 kiloNewtons (“kN”) or less, about 40 kN or less, about 35 kN or less, about 30 kN or less, about 25 kN or less, or about 20 kN or less. While a higher compression force can be used, this can retard penetration of saliva into the dosage forms, resulting in unacceptable disintegration rates. Compression processes suitable for use with the present invention are “low pressure” processes (e.g., a compression pressure of about 50 kN or less), and result in a solid pharmaceutical dosage form having a porous morphology. For example, a non-effervescent, orally disintegrating solid pharmaceutical dosage form of the present invention can be compressed using 500 mg of a powdered mixture in a 16/32″ die cavity and fitted with 16/32″ flat-faced bewel-edged punches using a CARVER® press autoseries tablet compression machine (Carver, Inc., Wabash, Ind.) at 25 kN pressure and 100% speed, or a STOKES® Single Station Press (Stokes-Merrill, Inc., Bristol, Pa.), or a KORSCH® XL-400 (Korsch AG, Berlin, Germany), or any other machine press known to those of ordinary skill in the art of tablet compression.

### Methods of Treatment

The present invention is also directed to methods of administering the non-effervescent, orally disintegrating solid pharmaceutical dosage forms of the present invention to a subject in need thereof (i.e., a subject suffering from a condition). The present invention includes methods of treatment comprising contacting the non-effervescent, orally disintegrating solid pharmaceutical dosage forms of the present invention with the buccal cavity of a subject suffering from, e.g., heartburn or another symptom associated with gastro-esophageal reflux disease.

For example, the present invention is directed to a method of treating gastro-esophageal reflux disease or a symptom thereof in a subject in need thereof, the method comprising administering a non-effervescent, orally disintegrating solid pharmaceutical dosage form, the dosage form comprising a compacted granulate-bead mixture comprising:

a bead portion comprising a lansoprazole spheroid having an enteric coating layer and a deformable coating layer thereon, wherein the deformable coating layer is about 10% to about 30% by weight of the bead portion and the bead portion has a D₅₀ of 400 μm or greater; and
a granulate portion comprising a disintegrant in a concentration of about 20% to about 60% by weight of the granulate,
wherein the bead portion and the granulate portion are present in a ratio of about 1:3 to about 3:1 and wherein upon oral administration without water the dosage form disintegrates in about 60 seconds or less.

As used herein, the term “subject” refers to any mammal, including humans and non-humans, such as, but not limited to, domestic and farm animals, zoo animals, sports animals, and pets.

As used herein, the term “administering to” refers to placing a pharmaceutical dosage form of the present invention in physical contact with the buccal cavity of a subject.

The terms “treat” and “treatment” refer to both therapeutic treatment and prophylactic, maintenance, or preventative measures, wherein the object is to prevent or slow down (lessen) an undesired physiological condition, disorder or disease, or obtain beneficial or desired clinical results. For purposes of this invention, beneficial or desired clinical results include, but are not limited to, alleviation of a symptom or a sign;

diminishment of extent of a condition, disorder or disease; stabilization (i.e., not worsening) of the state of condition, disorder or disease; delay in onset or slowing of condition, disorder or disease progression; amelioration of a condition, disorder or disease state, remission (whether partial or total), whether detectable or undetectable; or enhancement or improvement of condition, disorder or disease. Treatment includes eliciting a clinically significant response, without excessive levels of side effects. Treatment also includes prolonging survival as compared to expected survival if not receiving treatment.

The orally disintegrating solid pharmaceutical dosage forms of the present invention can be administered alone or in conjunction with other medications or pharmaceutical compositions. In some embodiments, the present invention is directed to a method of treating and or preventing diseases in a human subject by administering the orally disintegrating solid pharmaceutical dosage forms of the present invention to the human subject.

The orally disintegrating solid pharmaceutical dosage forms of the present invention are administered to the buccal cavity of a subject, where the dosage forms disintegrate, and the resulting particulate and/or liquid compositions can be swallowed by a subject.

Pharmaceutical dosage forms of the present invention contain a therapeutically effective amount of lansoprazole. The term “therapeutically effective amount” refers to an amount of lansoprazole that diminishes one or more symptoms of a disease or disorder (i.e., treats a disease or disorder) in a subject. For example, a therapeutically effective amount for the treatment of heartburn and other symptoms associated with GERD refers to an amount which, when administered, diminishes one or more symptoms associated with these disorders. Symptoms treated by lansoprazole effectively include heartburn and other symptoms associated with GERD.

The precise therapeutic dosage of lansoprazole necessary to be therapeutically effective can vary between subjects (e.g., due to age, body weight, sex, condition of the subject, the nature and severity of the disorder or disease to be treated, and the like). Thus, the therapeutically effective amount cannot be specified in advance and can be determined by a caregiver, for example, by a physician using, for example, dose titration. Appropriate therapeutically effective amounts can also be determined by routine experimentation using, for example, animal models.

The non-effervescent, orally disintegrating solid pharmaceutical dosage forms of the present invention have controlled bioavailability. Not being bound by any particular theory, the bioavailability of lansoprazole contained within the pharmaceutical dosage forms of the present invention is attributable to the protection of lansoprazole from degradation within the low-pH environment of the stomach. In some embodiments, the pharmaceutical dosage forms of the present invention have a bioavailability higher than other oral dosage forms containing a substantially equivalent amount of lansoprazole (e.g., about 2 times to about 5 times greater). In some embodiments, the pharmaceutical dosage forms of the present invention have a bioavailability substantially similar to a delayed-release capsule, suspension, or orally disintegrating tablet dosage form (e.g., PREVACID®) that contains a substantially equivalent dosage of lansoprazole.

In some embodiments, the present invention is directed to a method of administering lansoprazole to a subject in need thereof, the method comprising administering to the subject in need thereof a pharmaceutical dosage form of the present invention.

In some embodiments, the dissolution of lansoprazole from the solid pharmaceutical dosage forms of the present invention can be related to pharmacokinetic parameters and/or the in vivo concentration of lansoprazole and its metabolites. The in vivo concentration of lansoprazole and its metabolites, as well as pharmacokinetic parameters can be determined by sampling the blood plasma of a subject after administration of the solid pharmaceutical dosage forms of the present invention. Pharmacokinetic parameters that can be measured include, but are not limited to, AUCₜ, AUC_{inf}, and ln(AUC_{LAST}).

As used herein, “AUCₜ” refers to the Area Under the Concentration time curve (i.e., plot of plasma concentration vs. time) after lansoprazole administration. The area is conveniently determined by the “trapezoidal rule”: the data points are connected by straight line segments, perpendiculars are erected from the abscissa to each data point, and the sum of the areas of the triangles and trapezoids so constructed is computed.

As used herein, “AUC_{inf}” refers to the Area Under the Concentration time curve, wherein the last concentration is extrapolated to baseline based on the rate constant for elimination.

As used herein, “ln(AUC_{LAST})” refers to the Area Under the Concentration time curve determined by plotting plasma concentration on a natural logarithmic scale, using the last measured plasma concentration as the end point.

As used herein, “IntraCV” refers to an intra-assay coefficient of variation, which is the standard deviation within a sample set divided by the mean value of the sample set, with the result reported as a percentage.

In some embodiments, the bioavailability of lansoprazole from the dosage forms of the present invention is substantially equivalent to that observed upon administration of PREVACID® delayed release oral capsule, suspension, or orally disintegrating tablet dosage forms at an equivalent dosage. For example, the dosage forms of the present invention can have an AUCₜ or AUC_{inf} which is within about 80% to about 120%, about 90% to about 110%, about 95% to about 105%, or about equivalent to that observed when PREVACID® delayed release oral capsule, suspension, or orally disintegrating tablet dosage forms are administered at an equivalent dosage.

Having generally described the invention, a further understanding can be obtained by reference to the examples provided herein. These examples are given for purposes of illustration only and are not intended to be limiting.

### EXAMPLES

### Example 1

Lansoprazole spheroids of the present invention were prepared by a process of the present invention using the materials listed in Table 1. Inert seeds (CELLETS® 200 microcrystalline cellulose spheres) were placed in a fluid bed coating apparatus, and an aqueous polymeric dispersion of lansoprazole, a binder (hydroxypropyl cellulose) and a stabilizer (magnesium carbonate) was sprayed onto the inert seeds and the coated seeds were dried. The coated seeds were then sized by passing through a #35 mesh screen and retained on a #60 mesh screen to provide lansoprazole spheroids having a mean diameter of about 370 μm.

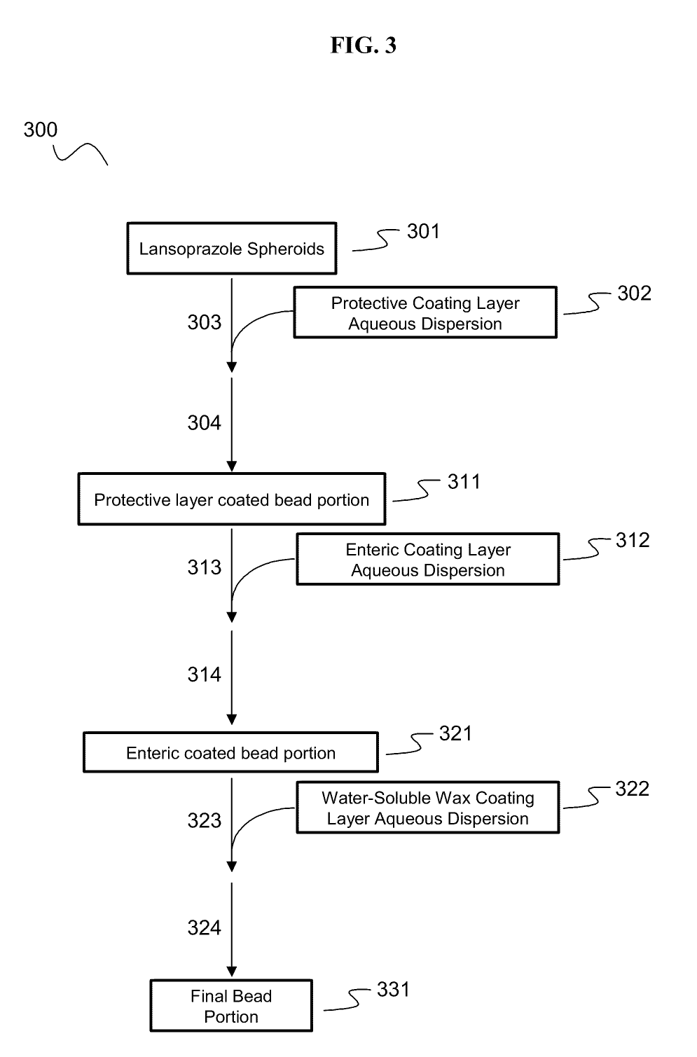

FIG. 3 provides a flow chart, 300, representing a process for preparing the bead portion of the orally disintegrating dosage forms using the excipients listed in Table 1. Referring to FIG. 3, the lansoprazole spheroids, 301, were placed in a fluid bed apparatus and a protective layer aqueous dispersion, 302, containing a polymer (hydroxypropylmethyl cellulose and hydroxypropyl cellulose), a binder (titanium dioxide) and a glidant (talc) was sprayed, 303, onto the lansoprazole spheroids and dried. These intermediate beads were then sized, 304, by passing through a #35 mesh screen and retained on a #60 mesh screen to provide a protective coating layer bead portion having a mean diameter of about 380 μm.

The protective coating layer bead portion, 311, was placed in a fluid bed apparatus and an enteric coating layer aqueous dispersion, 312, containing an enteric polymer (an anionic co-polymer of methacrylic acid and ethylacrylate, and a co-polymer of ethylacrylate and methyl methacrylate) and a plasticizer (propylene glycol) was sprayed, 313, onto the protective coating layer bead portion and dried. These intermediate beads were then sized, 314, by passing through a #30 mesh screen and retained on a #60 mesh screen to provide an enteric coated bead portion having a mean diameter of about 510 μm.

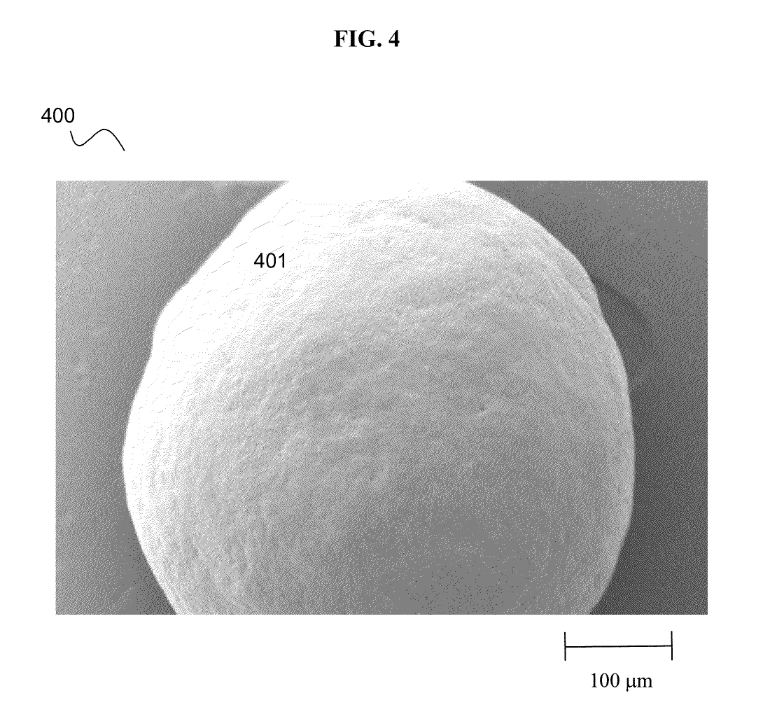

FIG. 4 provides a digital image of a scanning electron micrograph, 400, of an enteric coated bead portion. Referring to FIG. 4, the image, 400, shows that the surface of the enteric coating layer, 401, is smooth and even.

The enteric coated bead portion, 321, was placed in a fluid bed apparatus and a deformable coating layer aqueous dispersion, 322, containing a water-soluble wax (polyethylene glycol), a binder (microcrystalline cellulose), a glidant (colloidal silicon dioxide), and a colorant (iron oxide) was sprayed, 323, onto the enteric coated bead portion and dried. These intermediate beads were then sized, 324, by passing through a #30 mesh screen and retained on a #60 mesh screen to provide a final bead portion of the present invention, 331, which had a mean diameter of about 520 μm.

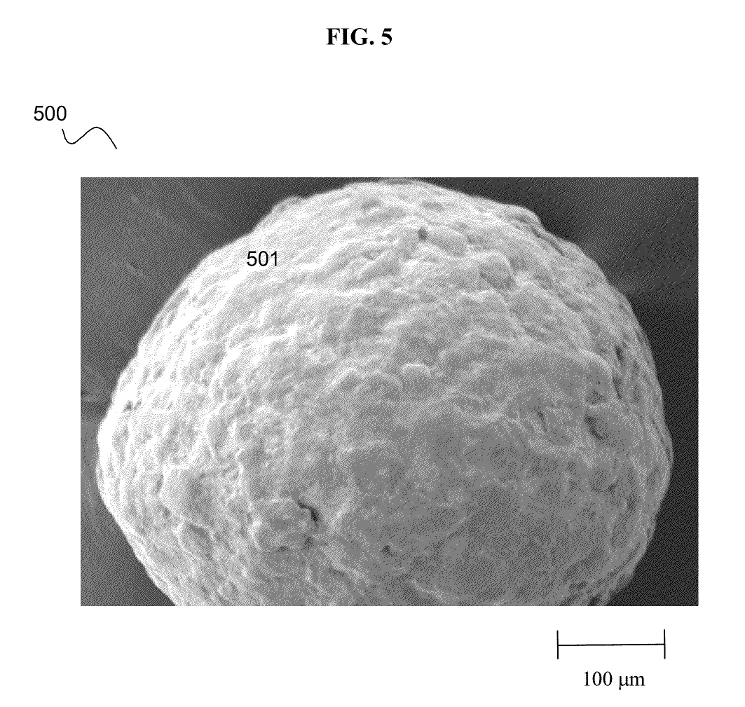

FIG. 5 provides a digital image of a scanning electron micrograph, 500, of a final bead of the present invention. Referring to FIG. 5, the image, 500, shows that the surface of the deformable coating layer, 501, is roughened and uneven.

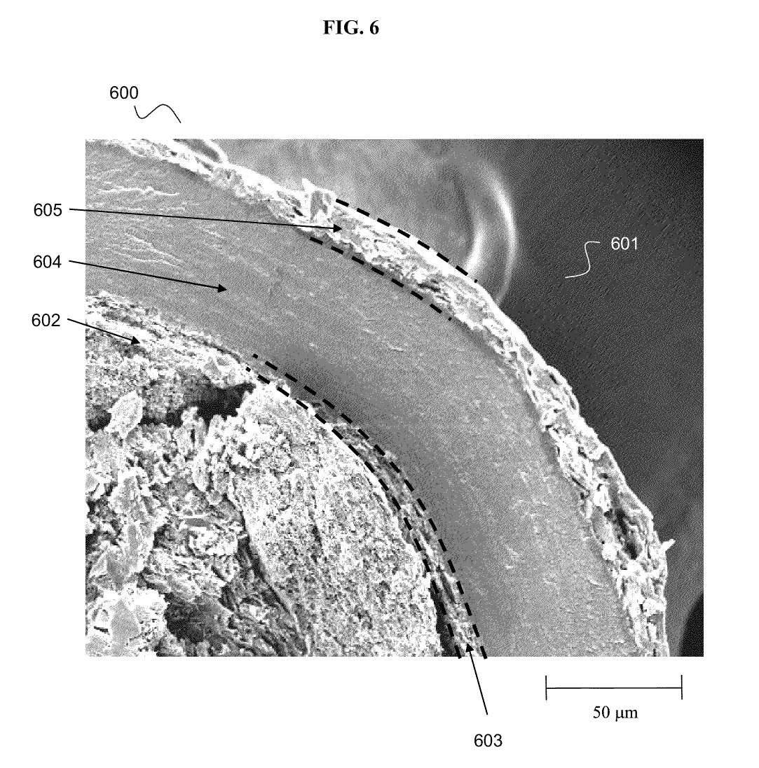

FIG. 6 provides a digital image of a scanning electron micrograph, 600, of a cross-sectional cutaway of a final bead, 601, of the present invention. Referring to FIG. 6, the image, 600, displays the lansoprazole spheroid, 602, the protective coating layer, 603, the enteric coating layer, 604, and the deformable coating layer, 605.

TABLE 1
Ingredients and their amounts used to prepare the bead portion
for a pharmaceutical dosage form of the present invention.

| | | |
|---|---|---|
| | 15 mg/ | 30 mg/ |
| Bead Portion Ingredients | Tablet | Tablet |

Lansoprazole spheroids

| | | |
|---|---|---|
| Lansoprazole, USP (Micronized) | 15 | 30 |
| Microcrystalline Cellulose Spheres (CELLETS ® 200) | 22.5 | 45 |
| Magnesium Carbonate, USP (Light Powder) | 7.5 | 15 |
| Hydroxypropyl Cellulose, NF (KLUCEL ® Type EF NF) | 2 | 4 |
| Hydroxypropyl Cellulose, NF (KLUCEL ® Type LF) | 2 | 4 |

Protective Coating Layer

| | | |
|---|---|---|
| Hypromellose 2910, USP (METHOCEL ® E5 Premium LV) | 3.5 | 7 |
| Hydroxypropyl Cellulose, NF (KLUCEL ® Type LF) | 1.5 | 3 |
| Titanium Dioxide, USP | 2.5 | 5 |
| Talc, USP | 2.5 | 5 |

Enteric Coating Layer

| | | |
|---|---|---|
| Methacrylic Acid Copolymer Dispersion, NF (EUDRAGIT ® L30D-55) | 45 | 90 |
| Aqueous Acrylic Polymer Dispersion (EUDRAGIT ® NE-30D) | 9 | 18 |
| Propylene Glycol, USP | 9 | 18 |

Deformable Coating Layer

| | | |
|---|---|---|
| Polyethylene Glycol, NF (3350 Powder) | 12 | 24 |
| Microcrystalline Cellulose, NF (AVICEL ® PH-105) | 11.95 | 23.9 |
| Colloidal Silicon Dioxide, NF (CAB-O-SIL ®) | 1.5 | 3 |
| Ferric oxide, NF (Synthetic Red Iron Oxide) | 0.05 | 0.1 |
| TOTAL | 147.5 mg | 295 mg |

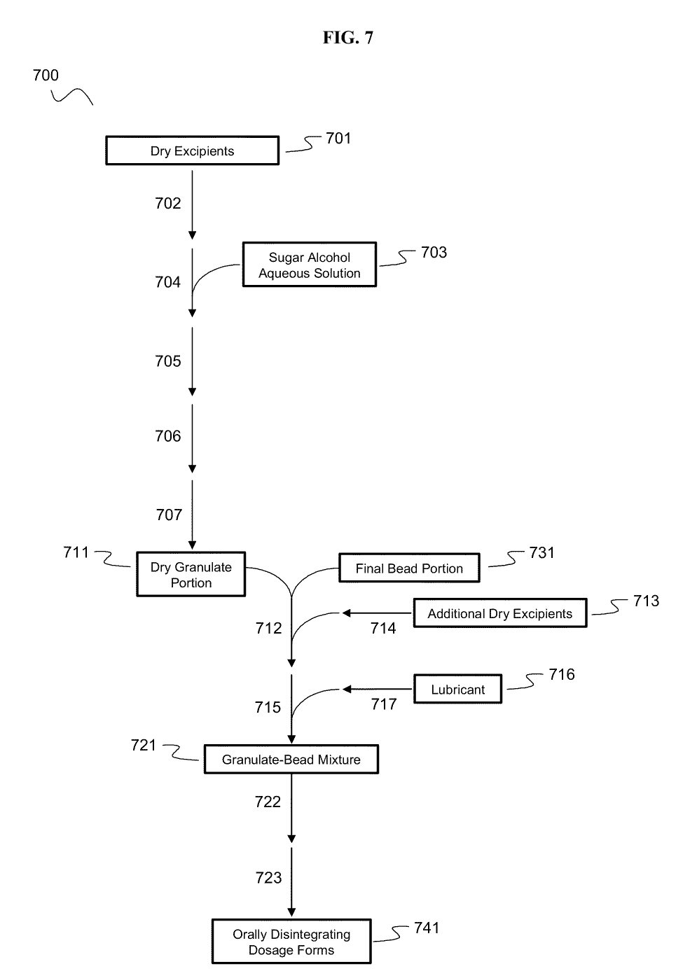

FIG. 7 provides a flow chart, 700, representing a process for preparing the granulate portion of the orally disintegrating dosage forms using the excipients listed in Table 2, as well as the process for forming the granulate-bead mixture to provide the orally disintegrating dosage forms. Referring to FIG. 7, dry excipients, 701, consisting of a super-disintegrant (crospovidone), a hydrophilic, water-insoluble excipient (microcrystalline cellulose), a sweetener (aspartame), and a glidant (colloidal silicon dioxide), were sized using a #20 mesh screen, 702, and placed in a COLLETTE200 600 mixer (Collette NV, Belgium). These dry excipients were then wet granulated, 704, with a sugar-alcohol aqueous solution, 703, comprising a sugar alcohol (mannitol). The resulting wet granulate was then wet milled, 705, using a QUADRO® co-mill (Quadro Engineering, Waterloo, Canada), dried, 706, to a dryness of about 3% or less water, by weight, and then milled a second time, 707, to form a dry granulate, 711. The dry granulate, 711, was mixed, 712, with the bead portion, 731, and additional dry excipients, 713, were added that were first sized, 714, using a #30 mesh screen. The resulting mixture was then lubricated, 715, by adding a lubricant (magnesium stearate), 716, that was first sized, 717, using a #30 mesh screen. The resulting granulate-bead mixture, 721, was compressed, 722, using a KORSCH® XL-400 (Korsch AG, Berlin, Germany), and then blister packaged, 723, to provide the orally disintegrating solid pharmaceutical dosage forms of the present invention, 741.

TABLE 2
Ingredients and their amounts used to prepare the granulate portion
for a pharmaceutical dosage form of the present invention.

| | | |
|---|---|---|
| | 15 mg/ | 30 mg/ |
| Granulation & Final Mixing | Tablet | Tablet |

Wet/Dry Granulate

| | | |
|---|---|---|
| Crospovidone, NF (POLYPLASDONE ® XL-10) | 62.5 | 125 |
| Microcrystalline cellulose, NF (AVICEL ® PH-101) | 50 | 100 |
| Aspartame powder, USP (NUTRASWEET ® Powder) | 4 | 8 |
| Colloidal silicon dioxide, NF (CAB-O-SIL ®) | 4.5 | 9 |
| Mannitol, USP (PARTECK ® M200) | 10 | 20 |
| Beads, 15 mg or 30 mg | 147.5 | 295 |

Additional Dry Granulate Excipients

| | | |
|---|---|---|
| Crospovidone, NF (POLYPLASDONE ® XL) | 15 | 30 |
| Aspartame powder, USP (NUTRASWEET ® Powder) | 2.5 | 5 |
| Strawberry Flavor (SN302419) | 1.75 | 3.5 |

Lubrication

| | | |
|---|---|---|
| Magnesium Stearate, NF | 2.25 | 4.5 |
| TOTAL | 300 mg | 600 mg |

### Example 2

The crush strength of various bead portions of the present invention was measured using a texture analyzer apparatus (TA.XT2i, Stable Microsystems) operated in compression mode. A P/6 (6 mm diameter) stainless steel probe was utilized for the tests at a trigger force of 1 g and a test speed of 0.05 mm/second. The pre-test and post-test speeds were 1 mm/second. The final bead portion prepared in Example 1 was tested along with lansoprazole pellets isolated from a PREVACID®30 mg orally disintegrating tablet. The data are listed in Table 3, which shows that the bead portions of the present invention exhibit superior crush strength. In particular, the final bead portion of the present invention has a crush strength about 210% greater than lansoprazole pellets of the prior art.

TABLE 3
Crush strength of various beads for use with the present
invention compared to a reference bead product.

| | Crush Strength |
|---|---|
| Bead Portion/Lansoprazole Spheroids | (n = 3) |
| Cellets ® 200 | 190 ± 40 g |
| Lansoprazole spheroids | 285 ± 50 g |
| Intermediate Bead A (Lansoprazole | 360 ± 60 g |
| spheroids + Protective Coating Layer) | |
| Intermediate Bead B (Lansoprazole spheroids + | 470 ± 80 g |
| Protective Coating Layer + Enteric Coating Layer) | |
| Final Bead Portion | 540 ± 100 g |
| Prevacid ® Pellets (30 mg) | 255 ± 30 g |

### Example 3

Placebo orally disintegrating solid pharmaceutical dosage forms were prepared by placing crospovidone (125 g), a sweetener (aspartame, 12 g), a flavorant (strawberry flavor, 3.5 g), and microcrystalline cellulose (Avicel PH-101, 110 g) in a COLLETTE® 600 mixer. The dry excipients were then sprayed with an aqueous solution of mannitol (19 g). The wet granulate was wet-milled, dried and dry-milled. The dry granulate was then administered to test subjects to evaluate the mouth feel of dry granulates as a function of the D₅₀ of the dry granulate. The data listed in Table 4 shows that mouth feel improved as the particle size of the dry granulate decreased. As a result, the particle size of dry granulate excipients was controlled. Specifically, the dry granulate utilized in Example 1 had a D₅₀ of 105 μm.

TABLE 4
Mouth feel as a function of dry granulate D₅₀.

| D₅₀ (μm) | Qualitative Mouth Feel |
|---|---|
| 634 | + |
| 429 | +++ |
| 409 | ++++ |
| 367 | +++++ |

Mouth feel was rated on a scale of least preferable (+) to most preferable (+++++).

### Example 4

Orally disintegrating solid pharmaceutical dosage forms were prepared by the process of Example 1 with various compression forces to provide orally disintegrating tablets having tablet hardness of about 1 kp to about 5 kp. The disintegration times for the various tablets were determined using a texture analyzer apparatus, the results of which are listed in Table 5. The acid resistance of the various dosage forms was determined using a USP Type II Apparatus containing 0.1 N HCl solution (500 mL) at a paddle speed of 75 rpm, the results of which are also listed in Table 5. The data demonstrates that acceptable disintegration times for the solid pharmaceutical dosage forms are obtained up to a tablet hardness of about 4 kp, and that all of the dosage forms of the present invention exhibit superior acid resistance.

TABLE 5
Average disintegration time and acid resistance for dosage forms
of the present invention as a function of tablet hardness.

| | Average Disintegration Time | Acid Resistance |
|---|---|---|
| Tablet Hardness | (n = 3) | (n = 3) |
| 1 kp | 4 s | 6% |
| 2 kp | 6 s | 6% |
| 3 kp | 9 s | 5% |
| 4 kp | 15 s | 5% |
| 5 kp | 68 s | 4% |

### Example 5

The stability of the orally disintegrating solid pharmaceutical dosage forms prepared according to Example 1 was tested using accelerated test conditions. The solid pharmaceutical dosage forms were placed in a controlled environment at 40° C. and 75% relative humidity. The concentration of N-oxide, sulfone and sulfide impurities was measured as function of time, the results of which are listed in Table 6.

TABLE 6
Dosage form stability as a function of time.

| | | | | Total | | |
|---|---|---|---|---|---|---|
| | N- | | | Im- | Lansoprazole | Disint. |
| Time | oxide | Sulfone | Sulfide | purities | Assay¹ | Time² |
| Initial | n/d | 0.18% | 0.02% | 0.21% | 98% | 8-12 s |
| (t = 0) | | | | | | |
| 7 days | n/d | 0.17% | 0.02% | 0.19% | — | — |
| 14 days | n/d | 0.15% | 0.01% | 0.22% | — | — |
| 1 month | n/d | 0.15% | 0.03% | 0.37% | 98% | 7-11 s |
| 2 months | n/d | 0.18% | 0.03% | 0.42% | 97% | 7-9 s |
| 3 months | n/d | 0.16% | 0.02% | 0.52% | 99% | 13-16 s |

¹Lansoprazole assay as a percentage of the labeled claim.
²Disintegration times were measured using a Disintegration Testing apparatus.
n/d = not detected.

### Example 6

The bioavailability of lansoprazole from the orally disintegrating solid pharmaceutical dosage forms of the present invention was measured in fasted subjects and compared to a reference product (PREVACID® 30 mg orally disintegrating tablets). The bioavailability data are listed in Table 7, and demonstrate that the orally disintegrating dosage forms of the present invention exhibit reliable bioavailability and are thus effective in treating GERD and other conditions of the gastrointestinal tract for which lansoprazole has been approved.

TABLE 7
Bioavailability of lansoprazole from orally disintegrating
solid pharmaceutical dosage forms of the present invention.

| | Lansoprazole | 90% | | |
|---|---|---|---|---|
| | (Ex. 1, | Confidence | | PREVACID ® |
| Parameter | 30 mg)¹ | Interval | IntraCV | ODT, 30 mg |
| Cₘₐₓ (ng/mL) | 1.016 | 0.96-1.075 | 26% | 1.0 |
| AUC₀₋ₜ (ng · | 1.034 | 1.00-1.067 | 14% | 1.0 |
| hr/mL) | | | | |
| AUC_{inf} (ng · | 1.034 | 1.00-1.067 | 14% | 1.0 |
| hr/mL) | | | | |

¹n = 109

### CONCLUSION

All of the various embodiments or options described herein can be combined in any and all variations. While the invention has been particularly shown and described with reference to some embodiments thereof, it will be understood by those skilled in the art that they have been presented by way of example only, and not limitation, and various changes in form and details can be made therein without departing from the spirit and scope of the invention. Thus, the breadth and scope of the present invention should not be limited by any of the above described exemplary embodiments, but should be defined only in accordance with the following claims and their equivalents.

All documents cited herein, including journal articles or abstracts, published or corresponding U.S. or foreign patent applications, issued or foreign patents, or any other documents, are each entirely incorporated by reference herein, including all data, tables, figures, and text presented in the cited documents.

What is claimed is:

1. A non-effervescent, orally disintegrating solid pharmaceutical dosage form comprising a compacted granulate-bead mixture, the granulate-bead mixture comprising:
a bead portion comprising a lansoprazole spheroid having an aspect ratio of about 0.75 to about 1.3 and having an enteric coating layer and a deformable coating layer thereon, wherein the deformable coating layer is about 10% to about 30% by weight of the bead portion and the bead portion has a D₅₀ of 400 μm or greater; and
a granulate portion comprising a disintegrant in a concentration of about 20% to about 60% by weight of the granulate,
wherein the bead portion and the granulate portion are present in a ratio of about 1:3 to about 3:1 and wherein upon oral administration without water the dosage form disintegrates in about 60 seconds or less.

2. The dosage form of claim 1, wherein the granulate portion comprises directly compressible mannitol in a concentration of about 1% to about 20% by weight of the granulate, and microcrystalline cellulose in a concentration of about 10% to about 40% by weight of the granulate.

3. The dosage form of claim 1, wherein the granulate portion comprises crospovidone in a concentration of about 20% to about 60% by weight of the granulate.

4. The dosage form of claim 1, wherein the granulate portion comprises directly compressible mannitol in a concentration of about 1% to about 10% by weight of the granulate.

5. The dosage form of claim 1, wherein the granulate portion comprises microcrystalline cellulose in a concentration of about 20% to about 40% by weight of the granulate.

6. The dosage form of claim 1, wherein the granulate portion comprises colloidal silicon dioxide in a concentration of about 10% or less by weight.

7. The dosage form of claim 1, wherein the granulate portion comprises a lubricant in a concentration of about 10% or less by weight.

8. The dosage form of claim 1, wherein the bead portion and the granulate portion are present in a ratio of about 1:2 to about 2:1 by weight.

9. The dosage form of claim 1, wherein the bead portion has a D₅₀ of 400 μm to about 550 μm.

10. The dosage form of claim 1, wherein the bead portion has a D₁₀ of about 350 μm or greater.

11. The dosage form of claim 1, wherein the bead portion has a D₉₀ of about 800 μm or less.

12. The dosage form of claim 1, wherein the granulate portion has a D₅₀ of about 50 μm to about 400 μm.

13. The dosage form of claim 1, wherein the bead portion comprises a lansoprazole spheroid having a protective coating layer thereon, wherein the lansoprazole spheroid and protective coating layer comprise a hydroxypropyl cellulose polymer having a molecular weight of about 95,000 Da or less.

14. The dosage form of claim 1, wherein the deformable coating layer comprises polyethylene glycol, microcrystalline cellulose, and a glidant.

15. The dosage form of claim 1, wherein the deformable coating layer is about 20% to about 30% by weight of the bead portion.

16. The dosage form of claim 1, wherein a surface of the deformable coating layer has a surface roughness of about 50 nm to about 10 μm.

17. The dosage form of claim 1, wherein the lansoprazole spheroid has an acid resistance of about 10% or less, as tested using a USP Type II Paddle Apparatus containing 475 mL of 0.1 N HCl at a paddle speed of 75 rpm.

18. The dosage form of claim 1, wherein the bead portion has a crush strength of about 300 g or more.

19. The dosage form of claim 1, wherein the solid pharmaceutical dosage form has a hardness of about 1 kp to about 5 kp.

20. A process to prepare a non-effervescent, orally disintegrating solid pharmaceutical dosage form, the process comprising:
(a) mixing a bead portion comprising lansoprazole spheroids having an aspect ratio of about 0.75 to about 1.3 and having an enteric coating layer and a deformable coating layer thereon, wherein the deformable coating layer is about 10% to about 30% by weight of the bead portion and the bead portion has a D₅₀ of 400 μm or greater with a granulate portion comprising a disintegrant in a concentration of about 20% to about 60% by weight of the granulate, wherein the bead portion and the granulate portion are present in a ratio of about 1:3 to about 3:1, to form a substantially homogeneous granulate-bead mixture; and
(b) compacting the granulate-bead mixture at a pressure of about 50 kN or less to provide the non-effervescent, orally disintegrating solid pharmaceutical dosage form, wherein upon oral administration without water the dosage form disintegrates in about 60 seconds or less.

21. The method of claim 20, wherein the granulate portion has a flow index of about 50 or more.

22. The method of claim 20, wherein a surface of the bead portion has a surface roughness of about 100 nm to about 10 μm.

23. A product prepared by the process of claim 20.

24. A method of treating gastro-esophageal reflux disease or a symptom thereof in a subject in need thereof, the method comprising administering a non-effervescent, orally disintegrating solid pharmaceutical dosage form, the dosage form comprising a compacted granulate-bead mixture comprising:
a bead portion comprising a lansoprazole spheroid having an aspect ratio of about 0.75 to about 1.3 and having an enteric coating layer and a deformable coating layer thereon, wherein the deformable coating layer is about 10% to about 30% by weight of the bead portion and the bead portion has a D₅₀ of 400 μm or greater; and
a granulate portion comprising a disintegrant in a concentration of about 20% to about 60% by weight of the granulate,
wherein the bead portion and the granulate portion are present in a ratio of about 1:3 to about 3:1 and wherein upon oral administration without water the dosage form disintegrates in about 60 seconds or less.
